(19) United States
(12) Patent Application Publication
(10) Pub. No.: US 2010/0129837 A1
(43) Pub. Date: May 27, 2010

(54) METHODS OF CAPTURING BACTERIAL WHOLE CELLS AND METHODS OF ANALYZING SAMPLES FOR BACTERIA

(76) Inventors: Patrick A. Mach, Shorewood, MN (US); Sridhar V. Dasaratha, Bangalore (IN); Jason W. Bjork, Newport, MN (US); Mara S. Reif-Wenner, Red Wing, MN (US); Chunmei Guo, Woodbury, MN (US); Heather M. Webb, Woodbury, MN (US); G. Marco Bommarito, Stillwater, MN (US); Brinda B. Badri, Woodbury, MN (US); Jörg Siekmöller, Mettmann (DE)

Correspondence Address: 3M INNOVATIVE PROPERTIES COMPANY, PO BOX 33427, ST. PAUL, MN, 55133-3427, US

(21) Appl. No.: 12/515,779

(22) PCT Filed: Nov. 20, 2007
(86) PCT No.: PCT/US07/85254

§ 371 Date: Dec. 15, 2009

Related U.S. Application Data
(60) Provisional application No. 60/867,016, filed on Nov. 22, 2006.
(60) Provisional application No. 60/867,098, filed on Nov. 22, 2006.

(51) Int. Cl. G01N 33/569; G01N 33/53

(52) U.S. Cl. 435/7.32; 435/7.1

(57) ABSTRACT

In certain embodiments, the invention relates to methods of capturing bacterial whole cells that includes the use of two or more antibodies having antigenic specificities for two or more distinct analytes characteristic of the specific bacterium. In certain embodiments, the invention relates to methods of analyzing a sample for a bacterium of interest. In particular, the methods are useful for detecting one or more analytes characteristic of a bacterium of interest, such as components of cell walls that are characteristic of a bacterium, particularly Staphylococcus aureus.

### RELATED APPLICATIONS

The present application claims the benefit of U.S. Provisional Patent Application Ser. Nos. 60/867,016 and 60/867,098, both filed on Nov. 22, 2006, and both of which are incorporated herein by reference in their entirety.

### GOVERNMENT RIGHTS

The U.S. Government may have certain rights to this invention under the terms of DAAD 13-03-C-0047 granted by the Department of Defense.

### BACKGROUND

The emergence of bacteria having resistance to commonly used antibiotics is an increasing problem with serious implications for the treatment of infected individuals. Accordingly, it is of increasing importance to determine the presence of such bacteria at an early stage and in a relatively rapid manner to gain better control over such bacteria. This also applies to a variety of other microbes.

One such microbe of significant interest is Staphylococcus aureus (“S. aureus”). This is a pathogen causing a wide spectrum of infections including: superficial lesions such as small skin abscesses and wound infections; systemic and life threatening conditions such as endocarditis, pneumonia and septicemia; as well as toxinoses such as food poisoning and toxic shock syndrome. Some strains (e.g., Methicillin-Resistant S. aureus) are resistant to all but a few select antibiotics.

Current techniques for the detection of microbes, particularly bacteria resistant to antibiotics, are generally time consuming and typically involve culturing the bacteria in pure form. One such technique for the identification of pathogenic staphylococci associated with acute infection, i.e., S. aureus in humans and animals and S. intermedius and S. hyicus in animals, is based on the microbe's ability to clot plasma. At least two different coagulase tests have been described: a tube test for free coagulase and a slide test for “cell bound coagulase” or clumping factor. The tube coagulase test typically involves mixing an overnight culture in brain heart infusion broth with reconstituted plasma, incubating the mixture for 4 hours and observing the tube for clot formation by slowly tilting the tube for clot formation. Incubation of the test overnight has been recommended for S. aureus since a small number of strains may require longer than 4 hours for clot formation. The slide coagulase test is typically faster and more economical; however, 10% to 15% of S. aureus strains may yield a negative result, which requires that the isolate be reexamined by the tube test.

Although methods of detecting S. aureus, as well as other microbes, have been described in the art, there would be advantage in improved methods of detection.

### SUMMARY

In certain embodiments, the invention provides methods for capturing whole bacterial cells. The methods involve the use of two or more antibodies having antigenic specificities for two or more distinct analytes characteristic of the specific bacterium. The two or more antibodies are preferably cooperative in their binding characteristics. That is, they are capable of simultaneously binding to distinct regions of the target analyte(s) or optimally are found to be of complementary binding whereby the binding of a distinct analyte is enhanced by the binding of another antibody.

In one such embodiment, the present invention provides methods of capturing an analyte characteristic of a specific bacterium, wherein the methods include: providing a sample suspected of including target whole cells comprising one or more analytes characteristic of a specific bacterium; providing two or more antibodies having antigenic specificities for two or more distinct analytes characteristic of the specific bacterium, wherein the antibodies comprise at least one monoclonal antibody; providing a solid support material; and providing contact between the sample, the solid support material, and the two or more antibodies under conditions effective to capture target whole cells with one or more analytes characteristic of a specific bacterium, if present.

In one preferred embodiment, the two or more antibodies are attached to the solid support material forming an analyte-binding material, and the method includes providing contact between the sample and the analyte-binding material under conditions effective to capture whole cells with one or more analytes characteristic of a specific bacterium, if present. Providing contact between the sample and the analyte-binding material can include simultaneous and/or sequential, preferably simultaneous, contact between the sample and the two or more antibodies.

In another preferred embodiment, providing contact between the sample, the solid support material, and the two or more antibodies includes providing contact between the two or more antibodies and the sample to form antibody-bound whole cells, and subsequently providing contact between the antibody-bound whole cells and the solid support material.

In certain embodiments, the invention provides methods of analyzing a sample for a microorganism of interest. In particular, the methods are useful for detecting one or more analytes characteristic of a microorganism (i.e., microbe) of interest, such as components of cell walls that are characteristic of a microbe, particularly Staphylococcus aureus.

In one embodiment, the present invention provides a method of analyzing an analyte characteristic of Staphylococcus aureus, wherein the method includes: providing a sample suspected of including target whole cells having one or more analytes characteristic of Staphylococcus aureus; providing two or more antibodies having antigenic specificities for two or more distinct analytes characteristic of Staphylococcus aureus, wherein the antibodies include at least one monoclonal antibody; providing a solid support material including magnetic particles; providing contact between the sample, the solid support material, and the two or more antibodies under conditions effective to capture target whole cells with one or more analytes characteristic of Staphylococcus aureus, if present; wherein the antibodies are selected from the group consisting of MAb-76, MAb-107, affinity-purified RxClf40, affinity-purified GxClf40, MAb 12-9, fragments thereof, and combinations thereof; and analyzing the captured target whole cells for the presence or absence of one or more analytes characteristic of Staphylococcus aureus.

In one embodiment, the present invention provides a method of analyzing a sample for a bacterium. The method includes: providing a sample suspected of including one or more analytes characteristic of a specific bacterium; providing two or more immobilized antibodies having antigenic specificities for two or more distinct analytes characteristic of the specific bacterium; providing two or more labeled antibodies having antigenic specificities for two or more distinct analytes characteristic of the specific bacterium, wherein the antibodies can be labeled with a direct or indirect enzymatic label; providing contact between the sample, the immobilized antibodies, and the labeled antibodies to bind the one or more analytes between the labeled antibodies and the immobilized antibodies; wherein, for each of the analytes present, the immobilized antibodies and the labeled antibodies comprise two or more antigen-binding pairs; and analyzing for the presence or absence of the specific bacterium.

In certain embodiments, providing contact between the sample, the immobilized antibodies, and the labeled antibodies includes: contacting the sample with the immobilized antibodies under conditions effective to capture one or more analytes characteristic of a specific bacterium, if present in the sample, to form one or more captured analytes; and contacting the one or more captured analytes, if present, with the labeled antibodies under conditions effective to cause binding between the one or more captured analytes and the labeled antibodies. Alternatively, providing contact between the sample, the immobilized antibodies, and the labeled antibodies includes: contacting the sample with the labeled antibodies under conditions effective to cause interaction between the one or more analytes characteristic of a specific bacterium, if present in the sample, and the labeled antibodies; and contacting the immobilized antibodies with the sample containing the labeled antibodies under conditions effective to cause binding between the labeled antibodies, the one or more analytes, and the immobilized antibodies.

Preferably, contacting the sample with the immobilized antibodies includes providing contact between the sample and each immobilized antibody simultaneously. Preferably, contacting the one or more captured analytes, if present, with the labeled antibodies includes providing contact between the captured analytes and each labeled antibody simultaneously.

The antibodies can be monoclonal, polyclonal, or a combination thereof. Preferably, they are selected from the group consisting of MAb-76, MAb-107, affinity-purified RxClf40, affinity-purified GxClf40, MAb 12-9, fragments thereof, and combinations thereof.

Preferably, the immobilized antibodies are bound to a solid support material, such as particulate material or the surface of a microwell plate, for example.

The antibodies can be labeled with a direct label (i.e., an enzyme) or indirect label (i.e., an enzyme-labeled secondary antibody, or an intermediate recognition chemistry, such as biotin, for an enzyme conjugate, such as streptavidin-bound enzyme). In certain embodiments, the antibodies are indirectly labeled with biotin, preferably biotin linked through a polyethylene oxide linker. If the antibody includes an indirect label such as this, the method can further include a step of reacting the labeled antibodies with an enzyme conjugate before, during, or after combining contacting the one or more captured analytes, if present, with the labeled antibodies.

The analysis can include analyzing colorimetrically, preferably using, for example, a chromogenic enzyme substrate that produces a color change upon interaction with the enzyme bound through the biotin to the labeled antibodies.

The analysis for the presence or absence of the specific bacterium can include quantifying the total amount of analyte present (and, thereby, quantifying the amount of bacterium present).

In certain embodiments, the invention provides methods that involve the use of an acousto-mechanical sensor. In one embodiment, the present invention provides a method of analyzing a sample for a bacterium, wherein the method includes: providing a sample suspected of including one or more analytes characteristic of a specific bacterium; providing two or more particle—antibody conjugates having antigenic specificities for two or more distinct analytes characteristic of the specific bacterium; providing a system comprising an acousto-mechanical sensor comprising a detection surface comprising two or more immobilized antibodies having antigenic specificities for two or more distinct analytes characteristic of the specific bacterium; providing contact between the sample, the immobilized antibodies on the detection surface of the acousto-mechanical sensor, and the particle-antibody conjugates to bind the one or more analytes between the particle-antibody conjugates and the immobilized antibodies; wherein, for each of the analytes present, the immobilized antibodies and the particle-antibody conjugates comprise two or more antigen-binding pairs; and analyzing for the presence or absence of the specific bacterium.

In certain such methods providing contact between the sample, the immobilized antibodies on the detection surface of the acousto-mechanical sensor, and the particle-antibody conjugates includes: contacting the sample with the particle-antibody conjugates, under conditions effective to cause interaction between the one or more analytes characteristic of the specific bacterium, if present in the sample, and the particle-antibody conjugates; and contacting the detection surface of the acousto-mechanical sensor with the sample containing the particle-antibody conjugates under conditions effective to cause binding between the particle-antibody conjugates, the one or more analytes, and the immobilized antibodies.

Alternatively, providing contact between the sample, the immobilized antibodies on the detection surface of the acousto-mechanical sensor, and the particle-antibody conjugates can include: contacting the sample with the immobilized antibodies under conditions effective to capture one or more analytes characteristic of a specific bacterium, if present in the sample, to form one or more captured analytes; and contacting the one or more captured analytes, if present, with the particle-antibody conjugates under conditions effective to cause binding between the one or more captured analytes and the particle-antibody conjugates.

In particularly preferred embodiments, the present invention provides a method that utilizes magnetic particles in the particle-antibody conjugates, and further includes: providing a magnetic field generator capable of providing a magnetic field proximate the detection surface that draws the target analyte with the attached magnetic particles to the detection surface of the sensor; selectively attaching the target biological analyte with the attached magnetic particles to the detection surface; disabling the magnetic field generator to substantially reduce the magnetic field proximate the detection surface; and operating the acousto-mechanical sensor to detect the attached target biological analyte while the detection surface is submersed in liquid. In certain embodiments, disabling of the magnetic field generator involves removing the magnetic field generator a sufficient distance to substantially reduce the magnetic field proximate the detection surface.

The analysis for the presence or absence of the specific bacterium can include quantifying the total amount of analyte present (and, thereby, quantifying the amount of bacterium present).

The present invention also provides an analyte-binding material that includes: a solid support material; antibodies MAb-76, MAb-107, affinity-purified RxClf40, affinity-purified GxClf40, MAb 12-9, fragments thereof, or combinations thereof, disposed on the solid support; and optionally a detectable marker. The solid support material can include particulate material. In certain embodiments, each particle of the particulate material has at least two antibodies that bind different analytes disposed thereon. The solid support material can be the surface of a microwell plate. In certain embodiments, a well of the microwell plate has immobilized therein a mixture of antibodies. Preferably, the microwell plate has at least two antibodies that bind to different analytes disposed thereon.

### DEFINITIONS

“Whole cell” means a biologically active bacterial cell that retains its structure during separation from other biological materials, but does not necessarily need to be able to reproduce.

The terms “analyte” and “antigen” are used interchangeably and refer to various molecules (e.g., Protein A) or epitopes of molecules (e.g., different binding sites of Protein A), or whole cells of the microorganism, that are characteristic of a microorganism (i.e., microbe) of interest. These include components of cell walls (e.g., cell-wall proteins such as protein A, and Clumping Factor, which is a cell wall-associated fibrinogen receptor that is found in S. aureus), external cell components (e.g., capsular polysaccharides and cell-wall carbohydrates), etc.

“Magnetic particles” means particles or particle agglomerates comprised of ferromagnetic, paramagnetic, or superparamagnetic particles, including dispersions of said particles in a polymer bead.

“Particle—antibody conjugates” refer to antibodies that are labeled with particulate material or particulate material that is labeled with one or more antibodies.

The term “enzyme-based” means a method that involves an enzyme for detection, as, for example, occurs in an enzyme-linked immunosorbent assay (ELISA).

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, an analyte-binding material that comprises “an” antibody can be interpreted to mean that the analyte-binding material includes “one or more” antibodies that bind different analytes.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

### I. Whole Cell Capture

The present invention is directed to various methods of capturing whole cells of a bacterium of interest from a sample based on the use of two or more analytes characteristic of the bacterium of interest. Specifically, the capture methods of the present invention include the use of two or more antibodies having antigenic specificities for two or more distinct analytes characteristic of the specific bacterium. The two or more antibodies are preferably cooperative in their binding characteristics. That is, they are capable of simultaneously binding to distinct regions of the target analyte(s) or optimally are found to be of complementary binding whereby the binding of a distinct analyte is enhanced by the binding of another antibody.

The capture methods for whole bacterial cells can be followed up by methods involving detecting the presence of an analyte on the whole cells characteristic of the bacterium of interest, and in certain embodiments, quantifying the bacterium of interest. Techniques of analyzing whole cells useful in methods of the present invention can be one of a wide variety of conventional techniques known to one of skill in the art. For example, such methods can include ELISA (e.g., colorimetric ELISA) or acousto-mechanical sensors, preferred embodiments of which are discussed in greater detail below.

The captured whole cells can also be lysed and used for detection of proteins, capsular and cell wall polysaccharides, DNA, RNA and other specific components of the target cells. The advantage of such an approach is that it selectively concentrates the cells and can provide better sensitivity and specificity. It also eliminates the inhibitors that may be present in the complex sample. For example, US Pat. App. Pub. No. 2004/0241824 discusses the use of anti-Protein A antibodies to adsorb the Staphylococcus aureus from a sample, separating the antibodies to which the S. aureus have been attached, lysing the S. aureus to extract the DNA followed by an assay to detect the DNA based on a polymerase chain reaction (PCR).

The present invention is advantageous in many situations where whole cell capture is part of the sample preparation step prior to detection or further use (e.g., analysis). It is known that the expression of a target protein can vary significantly for a given strain of bacteria. In these situations a single antibody against a single antigen of the targeted bacteria can result in some strains of the bacteria showing poor capture efficiency or not being captured at all. For these strains the sample preparation step would result in highly reduced availability of the bacteria for detection. As a result, this will increase the number of false negatives for the detection technique and this also has an adverse effect on the detection limit of the assay. By having a mix of particles coated with different antibodies or having different antibodies coated on the same particle, it is possible to increase the capture efficiency of bacterial strains showing poor or no capture with a single antibody. Thus, the assay sensitivity as well as the detection limit of an assay using whole cell capture can be improved by using the method of this invention. It has also been found that this can be achieved without negatively impacting the capture of those strains that are well captured with a single antibody technique.

Preferably, relatively small volumes of test sample can be used. Although test sample volumes significantly greater than 2 milliliters (mL) may be utilized, test samples on the order of 500 microliters (μL) are typically sufficient for methods of the present invention, although smaller sample sizes may be possible.

Preferably, using methods of the present invention, the capture time can be relatively short. For example, the capture time can be less than 30 minutes, less than 15 minutes, less than 5 minutes, less than 60 seconds, and even as short as 30 seconds.

Bacteria of particular interest include Gram positive and Gram negative bacteria. Particularly relevant organisms include members of the family Enterobacteriaceae, or the family Micrococcaceae or the genera Staphylococcus spp., Streptococcus spp., Pseudomonas spp., Enterococcus spp., Salmonella spp., Legionella spp., Shigella spp. Yersinia spp., Enterobacter spp., Escherichia spp., Bacillus spp., Listeria spp., Vibrio spp., Corynebacteria spp. as well as herpes virus, Aspergillus spp., Fusarium spp., and Candida spp. Particularly virulent organisms include Staphylococcus aureus (including resistant strains such as Methicillin Resistant Staphylococcus aureus (MRSA)), S. epidermidis, Streptococcus pneumoniae, S. agalactiae, S. pyogenes, Enterococcus faecalis, Vancomycin Resistant Enterococcus (VRE), Vancomycin Resistant Staphylococcus aureus (VRSA), Vancomycin Intermediate-resistant Staphylococcus aureus (VISA), Bacillus anthracis, Pseudomonas aeruginosa, Escherichia coli, Aspergillus niger, A. fumigatus, A. clavatus, Fusarium solani, F. oxysporum, F. chlamydosporum, Listeria monocytogenes, Listeria ivanovii, Vibrio cholera, V. parahemolyticus, Salmonella cholerasuis, S. typhi, S. typhimurium, Candida albicans, C. glabrata, C. krusei, Enterobacter sakazakii, E. coli O157 and multiple drug resistant Gram negative rods (MDR).

Of particular interest are Gram positive bacteria, such as Staphylococcus aureus. Typically, these can be detected by detecting the presence of a cell-wall component characteristic of the bacteria, such as a cell-wall protein. Also, of particular interest are antibiotic resistant microbes including MRSA, VRSA, VISA, VRE, and MDR. Typically, these can be detected by additionally detecting the presence of an internal cell component, such as a membrane protein, transport protein, enzyme, etc., responsible for antibiotic resistance.

Methods of the present invention could be used to capture whole bacterial cells from a sample using separate molecules (e.g., molecules like protein A and Clumping Factor for analysis of Staphylococcus aureus) or two different epitopes of the same molecule (e.g., a protein). Such analytes include, for example, cell-wall proteins such as protein A and microbial surface components recognizing adhesive matrix molecules (MSCRAMMs) such as fibrinogen-binding proteins (e.g., clumping factors), fibronectin-binding proteins, collagen-binding proteins, heparin-related polysaccharides binding proteins, and the like. Protein A and clumping factors, such as fibrinogen-binding factors and clumping factors A, B, and Efb, are also particularly useful in methods of detecting the presence of Staphylococcus aureus. Other cell-wall components of interest include capsular polysaccharides and cell-wall carbohydrates (e.g., teichoic acid and lipoteichoic acid).

Species of interest can be analyzed in a test sample that may be derived from a wide variety of sources, such as a physiological fluid, e.g., blood, saliva, ocular lens fluid, synovial fluid, cerebral spinal fluid, pus, sweat, exudate, urine, mucus, mucosal tissue (e.g., buccal, gingival, nasal, ocular, tracheal, bronchial, gastrointestinal, rectal, urethral, ureteral, vaginal, cervical, and uterine mucosal membranes), lactation milk, feces, or the like. Further, the test sample may be derived from a body site, e.g., wound, skin, anterior nares, nasopharyngeal cavity, nasal cavities, anterior nasal vestibule, scalp, nails, outer ear, middle ear, mouth, rectum, vagina, axilla, perineum, anus, rectum, or other similar site.

Besides physiological fluids, other test samples may include other liquids as well as solid(s) dissolved in a liquid medium. Samples of interest may include process streams, water, soil, plants or other vegetation, air, surfaces (e.g., contaminated), and the like.

The art describes various patient sampling techniques for the detection of bacteria, such as S. aureus. Such sampling techniques are suitable for the methods of the present invention as well. For example, it is common to obtain a sample from wiping the nares of a patient, e.g., patient's anterior nares, by swabbing with a sterile swab or sampling device. For example, one swab is used to sample each subject, i.e., one swab for both nares. The sampling can be performed, for example, by inserting the swab dry or pre-moistened with an appropriate solution into the anterior tip of the subject's nares and rotating the swab for two complete revolutions along the nares' mucosal surface.

A wide variety of swabs or other sample collection devices are commercially available, for example, from Puritan Medical Products Co. LLC, Guilford, Me., under the trade designation PURE-WRAPS, or from Copan Diagnostics, Inc., Murrietta, Calif., under the trade designations microRheologics nylon flocked swab and ESwab Collection and Transport System. A sample collection means such as that disclosed, for example, in U.S. Pat. No. 5,879,635 (Nason) can also be used if desired. Swabs can be of a variety of materials including cotton, rayon, calcium alginate, Dacron, polyester, nylon, polyurethane, and the like.

The sample collection device (e.g., swab) can then be cultured directly, analyzed directly, or extracted (e.g., by washing, elution by vortexing) with an appropriate solution, as discussed above for whole cell capture. Such extraction (i.e., elution) solutions typically include water and can optionally include a buffer and at least one surfactant. An example of an elution buffer includes, for example, phosphate buffered saline (PBS), which can be used in combination, for example, with TWEEN 20 (polyoxyethylene sorbitan monolaurate, available from Sigma-Aldrich Corp.) or PLURONIC L64 (poly(oxyethylene-co-oxypropylene) block copolymer, available from BASF Corp.). Other extraction solutions function to maintain specimen stability during transport from sample collection site to sample analysis sites. Examples of these types of extraction solutions include Amies' and Stuart's transport media.

The test sample (e.g., liquid) may be subjected to treatment prior to further analysis. This includes concentration, precipitation, filtration, centrifugation, distillation, dialysis, dilution, inactivation of natural components, addition of reagents, chemical treatment, etc.

The sample is contacted with appropriate reactant molecules for analyte binding (e.g., an analyte-binding material that includes a bacteria-recognizing reagent). Such reactant molecules include antibodies and optionally other reactant molecules such as lectins, enzymes, and receptors and other binding pair technologies, as well as other reactant molecules that recognize metabolic by-products (e.g., pH changes, detectable enzyme production). For example, in one embodiment, the sample can be contacted with one or more antibodies. Such antibodies can be attached to particulate material, a membrane, or other solid support material. Such analyte-binding materials are described in greater detail herein. Particularly preferred reactant molecules are those that are capable of direct interaction with target whole cells, particularly antibodies to whole cell surface antigens, lectins, and other proteins known to interact with whole cell surfaces.

Analyte-binding material useful in methods of the present invention for capture of the target analytes (e.g., target whole cells) typically includes a solid support material derivatized by coupling (non-covalently or covalently) to the support a reactant molecule that binds the target analytes. A mixture containing the target analytes (e.g., target whole cells) is contacted with the derivatized support to bind the target analytes to the biologically active substance, and unbound remaining mixture is removed from the support. Bound cells may be eluted from the support to obtain purified target analytes or processed while attached to the solid support material.

As mentioned above, the target analytes on whole cells can be detected by a reactant molecule (e.g., an S. aureus reactant molecule or a bacteria-recognizing reagent for S. aureus). In some embodiments, one or more antibodies, such as an S. aureus antibody, are employed as an S. aureus reactant. “S. aureus antibody” refers to an immunoglobulin having the capacity to specifically bind a given antigen inclusive of antigen binding fragments thereof. The term “antibody” is intended to include whole antibodies of a wide variety of isotypes (e.g., IgG, IgA, IgM, IgE, etc.), and fragments thereof from vertebrate, e.g., mammalian species which are also specifically reactive with foreign compounds, e.g., proteins. The antibodies can be monoclonal, polyclonal, or combinations thereof.

Antibodies can be fragmented using conventional techniques and the fragments screened for utility in the same manner as whole antibodies. Thus, the term includes segments of proteolytically cleaved or recombinantly prepared portions of an antibody molecule that are capable of selectively reacting with a certain protein. Non-limiting examples of such proteolytic and/or recombinant fragments include Fab, F(ab′)₂, Fv, and single chain antibodies (scFv) containing a VL and/or VH domain joined by a peptide linker. The scFv's can be covalently or non-covalently linked to form antibodies having two or more binding sites. Antibodies can be labeled with a wide variety of detectable moieties (i.e., detectable markers) known to one skilled in the art. In some aspects, the antibody that binds to an analyte one wishes to measure (the primary antibody) is not labeled, but is instead detected indirectly by binding of a labeled secondary antibody or other reagent that specifically binds to the primary antibody.

Various S. aureus antibodies are known in the art. For example, S. aureus antibodies are commercially available from Sigma-Aldrich and Accurate Chemical. Further, other S. aureus antibodies, such as the monoclonal antibody Mab 12-9, are described in U.S. Pat. No. 6,979,446. In certain preferred embodiments, an antibody is selected from those described herein (e.g., selected from the group consisting of MAb-76, MAb-107, affinity-purified RxClf40, affinity-purified GxClf40, MAb 12-9), fragments thereof, and combinations thereof. Such antibodies are also disclosed in U.S. patent application Ser. No. 11/562,759, filed on Nov. 22, 2006, and PCT App. Ser. No. U.S. Ser. No. 07/84,736, both entitled “ANTIBODY WITH PROTEIN A SELECTIVITY,” and in U.S. patent application Ser. No. 11/562,747, filed on Nov. 22, 2006, and PCT App. Ser. No. U.S. Ser. No. 07/84,739, both entitled “ANTIBODY WITH PROTEIN A SELECTIVITY,” and in U.S. Pat. App. Ser. No. 60/867,089, filed on Nov. 22, 2006 and U.S. patent application Ser. No. ______ (Attorney Docket No. 62611US005), filed on even date herewith, both of which are entitled “SPECIFIC ANTIBODY SELECTION BY SELECTIVE ELUTION CONDITIONS.”

Preferred antibodies are monoclonal antibodies. Particularly preferred are monoclonal antibodies that bind to Protein A of Staphylococcus aureus (also referred to herein as “S. aureus” or “Staph A”).

More particularly, in one embodiment, suitable monoclonal antibodies, and antigen binding fragments thereof, are those that demonstrate immunological binding characteristics of monoclonal antibody 76 as produced by hybridoma cell line 358A76.1. Murine monoclonal antibody 76 is a murine IgG2A, kappa antibody isolated from a mouse immunized with Protein A. In accordance with the Budapest Treaty, hybridoma 358A76.1, which produces monoclonal antibody 76, was deposited on Oct. 18, 2006 in the American Type Culture Collection (ATCC) Depository, 10801 University Boulevard, Manassas, Va. 20110-2209, and was given Patent Deposit Designation PTA-7938 (also referred to herein as accession number PTA-7938). The hybridoma 358A76.1 produces an antibody referred to herein as “Mab 76.” Mab 76 is also referred to herein as “Mab76,” “Mab-76,” “MAb-76,” “monoclonal 76,” “monoclonal antibody 76,” “76,” “M76,” or “M 76,” and all are used interchangeably herein to refer to immunoglobulin produced by hybridoma cell line 358A76.1 as deposited with the American Type Culture Collection (ATCC) on Oct. 18, 2006, and assigned Accession No. PTA-7938.

In another embodiment, suitable monoclonal antibodies, and antigen binding fragments thereof, are those that demonstrate immunological binding characteristics of monoclonal antibody 107 as produced by hybridoma cell line 358A107.2. Murine monoclonal antibody 107 is a murine IgG2A, kappa antibody isolated from a mouse immunized with Protein A. In accordance with the Budapest Treaty, hybridoma 358A107.2, which produces monoclonal antibody 107, was deposited on Oct. 18, 2006 in the American Type Culture Collection (ATCC) Depository, 10801 University Boulevard, Manassas, Va. 20110-2209, and was given Patent Deposit Designation PTA-7937 (also referred to herein as accession number PTA-7937). The hybridoma 358A107.2 produces an antibody referred to herein as “Mab 107.” Mab 107 is also referred to herein as “Mab107,” “Mab-107,” “MAb-107,” “monoclonal 107,” “monoclonal antibody 107,” “107,” “M107,” or “M 107,” and all are used interchangeably herein to refer to immunoglobulin produced by the hybridoma cell line as deposited with the American Type Culture Collection (ATCC) on Oct. 18, 2006, and given Accession No. PTA-7937.

Suitable monoclonal antibodies are also those that inhibit the binding of monoclonal antibody MAb-76 to Protein A of S. aureus. The present invention can utilize monoclonal antibodies that bind to the same epitope of Protein A of S. aureus that is recognized by monoclonal antibody MAb-76. Methods for determining if a monoclonal antibody inhibits the binding of monoclonal antibody MAb-76 to Protein A of S. aureus and determining if a monoclonal antibody binds to the same epitope of Protein A of S. aureus that is recognized by monoclonal antibody MAb-76 are well known to those skilled in the art of immunology.

Suitable monoclonal antibodies are also those that inhibit the binding of monoclonal antibody MAb-107 to Protein A of S. aureus. The present invention can utilize monoclonal antibodies that bind to the same epitope of Protein A of S. aureus that is recognized by monoclonal antibody MAb-107. Methods for determining if a monoclonal antibody inhibits the binding of monoclonal antibody MAb-107 to Protein A of S. aureus and determining if a monoclonal antibody binds to the same epitope of Protein A of S. aureus that is recognized by monoclonal antibody MAb-107 are well known to those skilled in the art of immunology.

Suitable monoclonal antibodies are those produced by progeny or derivatives of this hybridoma and monoclonal antibodies produced by equivalent or similar hybridomas.

Also included in the present invention include various antibody fragments, also referred to as antigen binding fragments, which include only a portion of an intact antibody, generally including an antigen binding site of the intact antibody and thus retaining the ability to bind antigen. Examples of antibody fragments include, for example, Fab, Fab′, Fd, Fd′, Fv, dAB, and F(ab′)₂ fragments produced by proteolytic digestion and/or reducing disulfide bridges and fragments produced from an Fab expression library. Such antibody fragments can be generated by techniques well known in the art.

Monoclonal antibodies useful in the present invention include, but are not limited to, humanized antibodies, chimeric antibodies, single chain antibodies, single-chain Fvs (scFv), disulfide-linked Fvs (sdFv), Fab fragments, F(ab′) fragments, F(ab′)₂ fragments, Fv fragments, diabodies, linear antibody fragments produced by a Fab expression library, fragments including either a VL or VH domain, intracellularly-made antibodies (i.e., intrabodies), and antigen-binding antibody fragments thereof.

Monoclonal antibodies useful in the present invention may be of a wide variety of isotypes. The monoclonal antibodies useful in the present invention may be, for example, murine IgM, IgG1, IgG2a, IgG2b, IgG3, IgA, IgD, or IgE. The monoclonal antibodies useful in the present invention may be, for example, human IgM, IgG1, IgG2, IgG3, IgG4, IgA1, IgA2, IgD, or IgE. In some embodiments, the monoclonal antibody may be murine IgG2a, IgG1, or IgG3. With the present invention, a given heavy chain may be paired with a light chain of either the kappa or the lambda form.

Monoclonal antibodies useful in the present invention can be produced by an animal (including, but not limited to, human, mouse, rat, rabbit, hamster, goat, horse, chicken, or turkey), chemically synthesized, or recombinantly expressed. Monoclonal antibodies useful in the present invention can be purified by a wide variety of methods known in the art for purification of an immunoglobulin molecule, for example, by chromatography (e.g., ion exchange, affinity, and sizing column chromatography), centrifugation, differential solubility, or by a wide variety of other standard techniques for the purification of proteins.

Suitable antibodies also include a high avidity anti-Staphylococcus aureus clumping factor protein polyclonal antibody preparation that detects recombinant clumping factor (rClf40) protein of S. aureus at a concentration of preferably at least 1 picogram per milliliter (pg/mL), and more preferably up to 100 pg/mL. Suitable antibodies also include a high avidity anti-Staphylococcus aureus clumping factor protein polyclonal antibody preparation demonstrating at least a 4-fold increase in detection sensitivity in comparison to a Staphylococcus aureus clumping factor protein antiserum.

In certain embodiments, a high avidity anti-Staphylococcus aureus clumping factor protein polyclonal antibody preparation is useful, wherein the high avidity anti-S. aureus clumping factor protein polyclonal antibody preparation is prepared by a method that includes obtaining antiserum from an animal immunized with recombinant clumping factor (rClf40) protein of S. aureus; binding the antiserum to an S. aureus clumping factor (CLf40) protein affinity column; washing the column with a wash buffer having 0.5 M salt and a pH of 4; and eluting the high avidity anti-S. aureus clumping factor protein polyclonal antibody preparation from the column with an elution buffer with a pH of 2. Herein, the high avidity anti-Staphylococcus aureus clumping factor polyclonal antibody preparations from rabbits and goats are referred to as affinity-purified RxClf40 and affinity-purified GxClf40, respectively. In some embodiments, the high avidity anti-Staphylococcus aureus clumping factor protein polyclonal antibody preparation may be obtained by a method that further includes enriching the antiserum for the IgG class of antibodies prior to binding the antiserum to an S. aureus clumping factor (Clf40) protein affinity column. Such enrichment may eliminate non-immunoglobulin proteins from the preparation and/or enrich for the IgG class of antibodies within the sample.

As used herein, antiserum refers to the blood from an immunized host animal from which the clotting proteins and red blood cells (RBCs) have been removed. An antiserum to a target antigen may be obtained by immunizing a wide variety of host animals. A wide variety of immunization protocols may be used.

Antibody avidity is a measure of the functional affinity of a preparation of polyclonal antibodies. Avidity is the compound affinity of multiple antibody/antigen interactions. That is, avidity is the apparent affinity of antigen/antibody binding, not the true affinity. Despite the heterogeneity of affinities in most antisera, one can characterize such populations by defining an average affinity (K₀).

Solid support materials can include particulate materials, membranes, gels (e.g., agarose), or other solid support materials such as the surfaces of tubes or plates. Exemplary solid supports can include materials such as nitrocellulose, polystyrene, polypropylene, nylon, ferromagnetic, paramagnetic, and superparamagnetic materials, gold sols, polycarbonate, polyethylene, cellulose, polysaccharide, and polyvinyl alcohol. For certain embodiments, particulate material and membranes are preferred.

Preferably, for certain embodiments, the analyte-binding material is particulate material (e.g., magnetic beads having an average particle size of less than 2 microns, and preferably, within a range of 0.05 micron to 1 micron). For example, magnetic beads functionalized with various groups such as carboxyl, amine, and tosyl are commercially available from various commercial sources such as Invitrogen (Carlsbad, Calif.) and Ademtech (Pessac, France). Streptavidin coated particles are also available from several sources such as Invitrogen (Carlsbad, Calif.), Ademtech (Pessac, France), and Miltenyi Biotec GmbH (Bergisch Gladbach, Germany).

The analyte-binding material preferably includes a solid support material having two or more antibodies disposed on the solid support, preferably particulate material. In certain embodiments, each particle of the particulate material has at least two antibodies that bind different analytes disposed thereon. For example, in certain embodiments, the analyte-binding material includes a solid support material (preferably particulate material) having antibodies MAb-107 and affinity-purified GxClf40 disposed thereon (preferably, in a ratio of 1:1).

Antibodies can be attached to a support material, preferably a particulate support material, through either covalent attachment or non-covalent attachment.

Non-covalent attachment of an antibody to a solid support material includes attachment by ionic interaction or hydrogen bonding, for example. One example of a non-covalent attachment included in the present invention is the well-know biotin-avidin system. Avidin-biotin affinity-based technology has found wide applicability in numerous fields of biology and biotechnology. The affinity constant between avidin and biotin is remarkably high (the dissociation constant, Kd, is approximately 10⁻¹⁵ M, see, Green, Biochem. J., 89, 599 (1963)) and is not significantly lessened when biotin is coupled to a wide variety of biomolecules. Numerous chemistries have been identified for coupling biomolecules to biotin with minimal or negligible loss in the activity or other desired characteristics of the biomolecule. A review of the biotin-avidin technology can be found in Applications of Avidin-Biotin Technology to Affinity-Based Separation, Bayer, et al., J. of Chromatography, pgs. 3-11 (1990).

Streptavidin, and its functional homolog avidin, are tetrameric proteins, having four identical subunits. Streptavidin is secreted by the actinobacterium, Streptomyces avidinii. A monomer of streptavidin or avidin contains one high-affinity binding site for the water-soluble vitamin biotin and a streptavidin or avidin tetramer binds four biotin molecules.

Biotin, also known as vitamin H or cis-hexahydro-2-oxo-1H-thieno-[3-4]-imidazole-4-pentanoic acid, is a basic vitamin which is essential for most organisms including bacteria and yeast. Biotin has a molecular weight of 244 daltons, much lower than its binding partners avidin and streptavidin. Biotin is also an enzyme cofactor of pyruvate carboxylase, trans-carboxylase, acetyl-CoA-carboxylase and beta-methylcrotonyl-CoA carboxylase which together carboxylate a wide variety of substrates.

Both streptavidin and avidin exhibit extremely tight and highly specific binding to biotin which is one of the strongest known non-covalent interactions between proteins and ligands, with a molar dissociation constant of 10⁻¹⁵ molar (M) (Green, Advances in Protein Chemistry, Vol. 29, pp. 85-133 (1975)), and a t1/2 of ligand dissociation of 89 days (Green, N.M., Advances in Protein Chemistry, Vol. 29, pp. 85-133 (1975)). The avidin-biotin bond is stable in serum and in the circulation (Wei et al., Experientia, Vol. 27, pp. 366-368 (1970)). Once formed, the avidin-biotin complex is unaffected by most extremes of pH, organic solvents, and denaturing conditions. Separation of streptavidin from biotin requires conditions, such as 8 M guanidine, pH 1.5, or autoclaving at 121° C. for 10 minutes (min).

Antibodies may be biotinylated using a wide variety of known methodologies. For example, antibodies may be biotinylated chemically, using activated biotin analogues, such as N-hydroxysuccinimidobiotin (NHS-biotin), which is commercially available from Pierce Chemical Company, Rockford, Ill., and requires the presence of a free primary amino group on the antibody.

In a preferred method of the present invention, magnetic particles can be coated with streptavidin and contacted with biotinylated antibodies. These particles can then be used for bacterial capture. With two or more antibodies, simultaneous or sequential capture can occur. Another option is that the biotinylated antibodies may be mixed with the sample to capture the bacteria and the antibody-bacteria complex can then be captured on the bead (Dynal T1 MyOne Streptavidin Package insert).

For certain embodiments, the ratio of the number of biotin molecules to the number of antibodies can be optimized to avoid aggregation for certain particles. For example, with the Ademtech 200-nm streptavidin-coated particles, a ratio of around 2:1 is preferred. Higher ratios, especially greater than 7:1 have shown aggregation issues for these particles.

Representative methods for covalent attaching an antibody to a particulate support material include utilizing functional groups in the support materials (such as carboxyl, amine, hydroxyl, maleimide, hydrazide) activated by activation compounds (such as glutaraldehyde, carbodiimide, cyanogen bromide) to react with another reactive groups (such as hydroxyl, amino, amido, or sulfhydryl groups) in an antibody. This bond may be, for example, a disulfide bond, thioester bond, amide bond, thioether bond, and the like. Antibodies may also be directly attached to support material functionalized with groups (such as tosyl, chloromethyl) that can directly react with a functional group on the antibody (such as amine).

Antibodies may be covalently bonded to a particulate support material by a wide variety of methods known in the art. For example, beads are commercially available that are derivatized with carboxyl groups. Antibodies can then be coupled to these beads through the formation of an amide linkage between a primary amine on the antibody and the carboxyl groups on the bead surface that is mediated by carbodiimide activation.

Typically, the particle concentration and antibody-to-particle ratios are optimized for the system of interest to achieve rapid capture. Generally, this is particle dependent. For example, for Dynal 1-μm particles the particle concentration is preferably greater than 0.04 mg/mL, more preferably greater than 0.1 mg/mL, and even more preferably greater than 0.16 mg/mL. For the same particles, the antibody to particle ratio is preferably greater than 1 μg/mg particles, more preferably greater than 10 μg/mL, and even more preferably greater than 40 μg/mg particles.

For example, for Ademtech 200-nm particles, the particle concentration is preferably greater than 0.04 mg/mL, more preferably greater than 0.1 mg/mL, and even more preferably greater than 0.16 mg/mL. For the same particles, the antibody to particle ratio is preferably at least 0.01 μg/mg particles, more preferably greater than 0.1 μg/mL, and even more preferably greater than 1 μg/mg particles. For the same particles, the antibody to particle ratio is preferably less than 10 μg/mg particles.

Suitable particles may or may not be blocked to prevent nonspecific binding. Such blocking may be done before or after antibody attachment. For example, certain magnetic beads (e.g., Dynal T1 MyOne streptavidin beads) are purchased blocked with bovine serum albumin (BSA). Other suitable blocking agents for nonspecific binding may be used, as is well known in the art.

Contact times (for example, mixing times) between the sample containing the target whole cells and the solid support material containing the antibodies can be no greater than 15 minutes, however as low as 30 seconds and as high as 30 minutes may be used. Such compositions may also include a buffer, such as PBS optionally with a PLURONIC L-64 surfactant, ethylenediamine tetraacetic acid (EDTA), BSA, or a combination thereof. Although physical agitation (or mixing) can be used for both large and small particles, the small particles may be used without mixing. Particles may be separated from the sample by settling, centrifugation, or filtration.

Preferably, magnetic particles are used and they are separated by the use of a magnetic field. Such separated particles (having whole cells thereon) can be washed with various buffers including, for example, PBS with PLURONIC L-64, or TWEEN 20, with or without BSA, etc.

Significantly, using whole capture methods of the present invention, preferably at least 20% of the target whole cells are captured, more preferably at least 50% of the target whole cells are captured, and even more preferably at least 80% of the target whole cells are captured.

### II Enzyme-Based Methods of Analysis

The present invention provides various enzyme-based methods of analyzing a sample for a bacterium of interest based on analysis of one or more analytes characteristic of the bacterium of interest using an enzymatic detection assay. Such methods can involve not only detecting the presence of one or more analytes characteristic of the bacterium of interest, but preferably identifying such analyte(s), which can lead to identifying a bacterium for which the analyte(s) is characteristic. In certain embodiments, analyzing the sample includes quantifying the total amount of analytes characteristic of the bacterium of interest.

Preferably, enzyme-based methods of the present invention include the use of two or more immobilized antibodies having antigenic specificities for two or more distinct analytes characteristic of the specific bacterium, the use of a second set of two or more labeled antibodies having antigenic specificities for two or more distinct analytes characteristic of the specific microorganism, wherein, for each of the analytes present, the immobilized antibodies and the labeled antibodies comprise two or more antigen-binding pairs. The antibodies of one set may be the same as the antibodies of the other set as long as they are capable of forming antigen-binding pairs.

Such antibodies are preferably cooperative in their binding characteristics. That is, they are capable of simultaneously binding to distinct regions of the target analyte(s) or optimally are found to be of complementary binding whereby the binding of their distinct analytes by one antibody set is enhanced by the binding of one or more other antibody sets.

Enzyme-based methods of the present invention include: providing a sample suspected of including one or more analytes characteristic of a specific bacterium; providing two or more immobilized antibodies having antigenic specificities for two or more distinct analytes characteristic of the specific bacterium; providing two or more labeled antibodies having antigenic specificities for two or more distinct analytes characteristic of the specific bacterium, wherein the antibodies can be labeled with a direct or indirect enzymatic label; providing contact between the sample, the immobilized antibodies, and the labeled antibodies to bind the one or more analytes between the labeled antibodies and the immobilized antibodies (i.e., a “sandwich”); wherein, for each of the analytes present, the immobilized antibodies and the labeled antibodies comprise two or more antigen-binding pairs; and analyzing for the presence or absence of the specific bacterium.

The two or more distinct analytes can be separate molecules, such as protein A and Clumping Factor, or two different epitopes of the same molecule. The labeled antibodies may include a solid phase label (e.g., particle—antibody conjugates) and/or may be immobilized on a solid support material (e.g., particulate material). The immobilized antibodies can be immobilized on a wide variety of solid support materials, such as particulate material, films, solid surfaces (e.g., the surface of a microwell plate), etc.

Enzyme-based methods of the present invention include immunoassays that involve contacting the sample with the immobilized antibodies under conditions effective to capture one or more analytes characteristic of a specific bacterium, if present in the sample, to form one or more captured analytes; and contacting the one or more captured analytes, if present, with the labeled antibodies under conditions effective to cause binding between the one or more captured analytes and the labeled antibodies. Alternatively, providing contact between the sample, the immobilized antibodies, and the labeled antibodies includes: contacting the sample with the labeled antibodies under conditions effective to cause interaction between the one or more analytes characteristic of a specific bacterium, if present in the sample, and the labeled antibodies; and contacting the immobilized antibodies with the sample containing the labeled antibodies under conditions effective to cause binding between the labeled antibodies, the one or more analytes, and the immobilized antibodies.

Conditions for capture and conditions for binding of one or more analytes between labeled antibodies and immobilized antibodies (and forming a “sandwich” assay) can be determined readily by one of skill in the art.

In certain preferred embodiments, contacting the sample with the immobilized antibodies includes providing contact between the sample and each immobilized antibody simultaneously, although sequential contact can also be used. Analogously, in certain preferred embodiments, contacting the one or more captured analytes, if present, with the labeled antibodies includes providing contact between the captured analytes and each labeled antibody simultaneously, although sequential contact can also be used.

Typically, analyzing for the presence or absence of the specific bacterium can occur through the presence of one or more analytes or the absence of all analytes. If desired, the analysis can involve quantification of the total amount of captured analytes. This can occur using standard curves and methodology well known to one of skill in the art.

Enzyme-based methods of the present invention involve enzyme-linked immunosorbent assays (ELISA's), particularly colorimetric ELISA's. The present invention is advantageous over conventional techniques for analyzing samples for such bacteria because of the improved time required to obtain a useful result, ease of use, performance (sensitivity, specificity, etc.), and conservative (broad specie coverage) detection, etc.

In such methods, significantly, relatively small volumes of test sample can be used. Although test sample volume as high as 2 milliliters (mL) may be utilized, advantageously test samples on the order of 10 microliters (μL) are sufficient for methods of the present invention, with 50-100 μL being preferred for certain embodiments.

Using preferred methods of the present invention, the initial capture time can be relatively short. Also, the detection time can be relatively short. For example, the detection time can be less than 60 minutes, less than 30 minutes, less than 15 minutes, less than 10 minutes, and even as short as 5 minutes. For a typical ELISA, the detection time is 2 to 4 hours.

Bacteria of particular interest include Gram positive bacteria and Gram negative bacteria as discussed above for whole cell capture. Of particular interest are Gram positive bacteria, such as Staphylococcus aureus. Typically, these can be detected by detecting the presence of a cell-wall component characteristic of the bacteria, such as a cell-wall protein. Also, of particular interest are antibiotic resistant microbes including MRSA, VRSA, VISA, VRE, and MDR. Typically, these can be detected by additionally detecting the presence of an internal cell component, such as a membrane protein, transport protein, enzyme, etc., responsible for antibiotic resistance.

The present invention is advantageous over conventional techniques for analyzing samples for such microbes because the signal for the analyte (e.g., external cell-associated components and/or cell markers from the internal portion of the cells) characteristic of the bacterium is enhanced. Methods of the present invention could be used to analyze a sample for separate molecules (e.g., molecules like protein A and Clumping Factor for analysis of Staphylococcus aureus) or two different epitopes of the same molecule (e.g., a protein). Such analytes may be present on fragments of cells or whole cells, as discussed above.

Such analytes include, for example, cell-wall proteins such as protein A and microbial surface components recognizing adhesive matrix molecules (MSCRAMMs) such as fibrinogen-binding proteins (e.g., Clumping Factor), fibronectin-binding proteins, collagen-binding proteins, heparin/heparin-related polysaccharides binding proteins, and the like. Protein A and Clumping Factor, such as fibrinogen-binding proteins and clumping factors A, B, and Efb, are also particularly useful in methods of detecting the presence of Staphylococcus aureus. Other external cell components of interest include capsular polysaccharides and cell-wall carbohydrates (e.g., teichoic acid and lipoteichoic acid).

If desired, methods of the present invention can further include analyzing the sample for an internal cell component, which may or may not be associated with a cell membrane, as the analyte of interest. Internal cell components are particularly useful in analyzing antibiotic resistant microbes, such as MRSA, VRSA, VISA, VRE, and MDR. Internal cell components that can be characteristic of such microbes include membrane proteins. Examples of such membrane proteins include cytoplasmic membrane proteins, inner membrane proteins, outer membrane proteins, and cell membrane proteins. Cytoplasmic membrane proteins, such as penicillin binding proteins (PBP) (e.g., PBP2′ or PBP2a) can be particularly characteristic of antibiotic resistant microbes. For example, the cytoplasmic membrane protein PBP2′ is characteristic of MRSA.

Species of interest can be analyzed in a test sample that may be derived from any of a wide variety of sources, such as a physiological fluid, as discussed above for whole cell capture. Further, the test sample may be derived from a wide variety of body sites, as discussed above for whole cell capture.

Samples of particular interest include mucus-containing samples, such as nasal samples (from, e.g., nares, nasopharyngeal cavity, nasal cavities, anterior nasal vestibule, etc.), as well as samples from the outer ear, middle ear, mouth, rectum, vagina, or other similar tissue. Examples of specific musosal tissues include buccal, gingival, nasal, ocular, tracheal, bronchial, gastrointestinal, rectal, urethral, ureteral, vaginal, cervical, and uterine mucosal membranes.

Besides physiological fluids, other test samples may include other liquids as well as solid(s) dissolved in a liquid medium, as discussed above for whole cell capture. Samples can also include cultured cells.

Various patient sampling techniques, as well as a wide variety of swabs or other sample collection devices, can be used, as discussed above for whole cell capture.

The sample collection device (e.g., swab) can then be cultured directly, analyzed directly, or extracted with an appropriate solution, as discussed above for whole cell capture.

The test sample (e.g., liquid) may be subjected to treatment prior to further analysis. This includes concentration, precipitation, filtration, centrifugation, distillation, dialysis, dilution, inactivation of natural components, addition of reagents, chemical treatment, etc.

That is, the test sample can be prepared using a wide variety of means well-known to those of skill in the art. For example, the sample could be disrupted to make available for analysis an analyte characteristic of the specific bacterium of interest using physical means (e.g., sonication, pressure, boiling or other heating means, vortexing with glass beads, etc.). Alternatively, the sample could be disrupted to make available for analysis an analyte characteristic of the specific microorganism of interest using various chemical reagents, which can include one or more components.

In certain embodiments, methods of the present invention include lysing the cells in the test sample, although whole cells are often desirable for analysis. In the methods of the present invention, lysing can include contacting the cells with a lysing agent or physically lysing the cells. Lysing can be conducted under conventional conditions, such as, for example, at a temperature of 5° C. to 42° C. (probably as high as 50° C.), preferably at a temperature of 15° C. to 25° C. Significantly, the lysing can occur using uncultured cells, i.e., a direct test sample, although cultured cells can be used as well.

Lysing can occur upon physically lysing the cells. Physical lysing can occur upon vortexing the test sample with glass beads, sonicating, heating and boiling, or subjecting the test sample to high pressure, such as occurs upon using a French press, for example

Lysing can also occur using a lysing agent. Suitable lysing agents include, for example, enzymes (e.g., protease, glycosidases, nucleases). Exemplary enzymes include lysostaphin, pepsin, glucosidase, galactosidase, lysozyme, achromopeptidase, endopeptidases, N-acetylmuramyl-L-alanine amidase, endo-beta-N-acethylglucosaminidase, ALE-1, DNase, and RNase. Various combinations of enzymes can be used if desired. Lysostaphin is particularly useful in methods of detecting the presence of Staphylococcus aureus.

Other lysing agents include salts (e.g., chaotrophic salts), solubilizing agents (e.g., detergents), reducing agents (e.g., beta-mercaptoethanol (BME), dithiothreitol (DTT), dithioerythritol (DTE), tris(2-carboxyethyl) phosphine hydrochloride (TCEP; Pierce Chemical Company, Rockford, Ill.), cysteine, n-acetyl cysteine), acids (e.g., HCl), and bases (e.g., NaOH). Such lysing agents may be more suitable for certain organisms than for others, for example, they can be more suitable for use with Gram negative bacteria than with Gram positive bacteria.

Various combinations of lysing agents and/or methods can be used if desired.

Methods of lysing are further discussed in U.S. Pat. App. Pub. No. 2005/0153370 A1. In particular, such lysing methods involve detecting one or more components of cell walls that are characteristic of a species of interest (e.g., a microbe of interest), and optionally one or more internal cell components that are further characteristic of a species of interest (e.g., an antibiotic resistant microbe of interest). It is believed that the cell-wall fragments analyzed are solid pieces of cell wall. That is, it is believed that they are not solubilized upon lysing; rather, the cell wall is merely broken into pieces. Furthermore, the cell-wall component that is analyzed is still part of (i.e., in or on) the cell wall fragments. That is, they are not solublized upon lysing. Significantly, this enhances the signal of the cell-wall component relative to the same component in an unlysed cell.

Additionally, if desired, and the sample is a mucus-containing sample, it can be further treated, either before or after lysing, with at least one reagent that can include a mucolytic agent. Treatment of mucus-containing samples with mucolytic agents can reduce the interference resulting from the presence of mucus during the analysis.

Examples of mucolytic agents include enzymes (e.g., pepsin, DNases, RNases, glucosidases, galactosidases, glycosidases), salts (e.g., chaotrophic salts), solubilizing agents (e.g., surfactants, detergents), reducing agents (e.g., beta-mercapto ethanol (BME), dithiotreotol (DTT), dithioerythritol (DTE), cysteine, TCEP, n-acetyl cysteine), and acids (e.g., HCl). Various combinations of such mucolytic agents can be used if desired. One of skill in the art will understand that there can be overlap between lysing agents and mucolytic agents; although not all lysing agents will be mucolytic, for example.

In certain embodiments, if the sample is a mucus-containing sample, and the mucolytic agent is a reducing agent, the reducing agent may be acidified (e.g., having a pH of less than 3). Reducing agents can be acidified using a variety of acids, such as inorganic acids (e.g., HCl) or organic acids (e.g., lactic acid, citric acid). Alternatively, if used in sufficiently high concentrations, the pH of the reducing agent does not need to be adjusted with an acid.

Typically, but optionally, after adding a reducing agent, the sample preparation involves inactivating the reducing agent in the composition. This can be done, for example, by providing a competitive substrate (for example, bovine serum albumen for n-acetyl cysteine). Other examples of reagents that inactivate the reducing agent include a diluent including a neutralizing buffer. Representative ingredients for neutralizing buffers can include, for example, buffering agent(s) (e.g., phosphate), salt(s) (e.g., NaCl), protein stabilizer(s) (e.g., BSA, casein, serum) polymer(s), saccharides, and/or detergent(s) or surfactant(s) (e.g., one or more of the following agents listed by tradenames and commonly available sources: NINATE 411 (amine alkylbenzene sulfonate, available from Stepan Co., Northfield, Ill.), ZONYL FSN 100 (Telomer B monoether with polyethylene glycol, available from E.I. DuPont de Nemours Co.), Aerosol OT 100% (sodium dioctylsulfosuccinate, available from American Cyanamide Co.), GEROPON T-77 (sodium N-oleyl-N-methyltaurate, available from Rhodia Novacare), BIO-TERGE AS-40 (sodium olefin (C₁₄-C₁₆)sulfonate, available from Stepan Co.), STANDAPOL ES-1 (sodium polyoxyethylene(1) laurylsulfate, available from Cognis Corp., Ambler, Pa.), TETRONIC 1307 (ethylenediamine alkoxylate block copolymer, available from BASF Corp.), SURFYNOL 465, 485, and 104 PG-50 (all available from Air Products and Chemicals, Inc.), IGEPAL CA210 (octylphenol ethoxylate, available from Stepan Co.), TRITON X-45, X-100, and X-305 (octylphenoxypolyethoxy ethanols, all available from The Dow Chemical Co.), SILWET L-7600 (polydimethylsiloxane methylethoxylate, available from Momentive Performance Materials, Inc., Wilton, Conn.), RHODASURF ON-870 (polyethoxylated(2) oleyl alcohol, available from Rhodia Novacare), CREMOPHOR EL (polyethyoxylated castor oil, available from BASF Corp.), TWEEN 20 and TWEEN 80 (polyoxyethylene sorbitan monolaurate and monooleate, both available from Sigma-Aldrich Corp.), BRIJ 35 (polyoxyethylene(23) dodecyl ether, available from Sigma-Aldrich Corp.), CHEMAL LA-9 (polyoxyethylene(9) lauryl alcohol, available from PCC Chemax, Piedmont, S.C.), PLURONIC L64 (poly(oxyethylene-co-oxypropylene) block copolymer, available from BASF Corp.), SURFACTANT 10G (p-nonylphenoxypoly(glycidol), available from Arch Chemicals Inc., Norwalk, Conn.), SPAN 60 (sorbitan monostearate, available from Sigma-Aldrich Corp.), CREMOPHOR EL (a polyethoxylated castor oil, available from Sigma-Aldrich Corp.)). If desired, the neutralizing buffer can also be used to adjust the pH of the sample.

In addition to, or alternative to, a reducing agent, the sample preparation of a mucus-containing sample can include the use of one or more surfactants or detergents (e.g., subsequently to or concurrently with, the combining of the sample and the enzymatic lysing agent with the mucolytic agent). Suitable surfactants can be nonionic, anionic, cationic, or zwitterionic. Suitable examples include sodium dodecyl sulfate (SDS) and sodium lauryl sulfate (SLS). Various combinations of surfactants can be used, if desired.

Optionally, the sample preparation method can include subsequently inactivating the surfactant. This can be done, for example, by providing a competitive substrate. Other examples of inactivating the surfactant include using reagent neutralizing buffers, such as a buffer that is sufficient to adjust the pH of the mucolytic test sample and surfactant to a pH of at least 5. Preferably, the buffer is sufficient to adjust the pH to no greater than 8.

Furthermore, if one or more of the sample preparation reagents is acidic, the subsequent composition including the analyte of interest is preferably neutralized to a pH of 7 to 7.5 or near 7.2. This can be done, for example, by providing a buffer and/or a diluent.

In certain embodiments, the sample is contacted with appropriate reactant molecules for analyte binding (e.g., an analyte-binding material that includes a bacterial-recognizing reagent). Such reactant molecules typically include antibodies. Such antibodies can be attached to particulate material, a membrane, or other solid support material.

As mentioned above for whole cell capture, the target analytes (i.e., analytes or components of interest), whether on whole cells or in lysed material, can be detected by a reactant molecule (e.g., an S. aureus reactant molecule or a bacteria-recognizing reagent for S. aureus). In some embodiments, one or more antibodies, such as an S. aureus antibody, are employed as an S. aureus reactant. “S. aureus antibody” refers to an immunoglobulin having the capacity to specifically bind a given antigen inclusive of antigen binding fragments thereof. The term “antibody” is intended to include whole antibodies of any isotype (IgG, IgA, IgM, IgE, etc.), and fragments thereof from vertebrate, e.g., mammalian species which are also specifically reactive with foreign compounds, e.g., proteins.

For methods of analysis (e.g., including capture and detecting) of the present invention, the antibodies can be monoclonal, polyclonal, or combinations thereof, as discussed above for whole cell capture. The discussion of antibodies for whole cell capture applies equally to methods of analysis, whether for whole cells or lysed cells, including capturing and detecting. Exemplary antibodies for methods of analysis are those discussed above for whole cell capture, including, for example, those selected from the group consisting of MAb-76, MAb-107, affinity-purified RxClf40, affinity-purified GxClf40, MAb 12-9), fragments thereof, and combinations thereof. Preferred antibodies are monoclonal antibodies. Particularly preferred are monoclonal antibodies that bind to Protein A of Staphylococcus aureus (also referred to herein as “S. aureus” or “Staph A”).

In certain preferred embodiments of the methods of analysis of the present invention, the two or more immobilized antibodies include two (isolated) monoclonal antibodies, particularly MAb12-9 and MAb-76. In certain preferred embodiments of the present invention, the two or more labeled antibodies include one monoclonal antibody, such as MAb-107, and one polyclonal antibody, such as affinity-purified RxClf40.

Immobilized antibodies are preferably immobilized on a solid support material. Suitable solid support materials can include particulate materials, membranes, gels (e.g., agarose, pvp), or other solid support materials such as the surfaces of tubes or plates (e.g., microwell plates). Exemplary solid supports can include materials such as nitrocellulose, polystyrene, polypropylene, nylon, gold sols, latex, and the like. For certain embodiments, particulate material and membranes are preferred.

The analyte-binding material includes a solid support material having one or more antibodies disposed on the solid support. In certain embodiments, the solid support material (e.g., each particle or each well of a microwell plate) has at least two antibodies that bind different analytes disposed thereon. For example, in certain embodiments, the analyte-binding material includes a solid support material having antibodies MAb-76 and MAb 12-9 disposed thereon (preferably, in a concentration combination of 1 μg/mL MAb-76 and 7.5 μg/mL MAb 12-9). Depending on the desired results and antibodies used, the concentrations and densities of immobilized antibodies can be varied by one of skill in the art.

Antibodies can be attached to a solid support material through either covalent attachment or non-covalent attachment.

Non-covalent attachment of an antibody to a solid support material includes attachment by passive adsorption and/or absorption, ionic interaction, or hydrogen bonding, for example.

Representative methods for covalent attaching an antibody to a particulate support material include chemical crosslinkers, such as heterobifunctional crosslinking compounds (i.e., “linkers”) that react to form a bond between reactive groups (such as hydroxyl, amino, amido, or sulfhydryl groups) in an antibody and other reactive groups (of a similar nature) in the support material. This bond may be, for example, a peptide bond, disulfide bond, thioester bond, amide bond, thioether bond, and the like.

Antibodies may be covalently bonded to a particulate support material by any of the methods known in the art. For example, glutaraldehyde, aldehyde-Schiffs base, n-hydroxyl succinimide, azlactone, cyanogen bromide, maleic anhydride, etc., may be used as attachment chemistries.

One example of a non-covalent labeling of antibodies included in the present invention is the well-known biotin-avidin system. Such a system can also be used in the labeling system of the antibodies for detection, particularly when enzymatic detection systems are used as in an ELISA.

Avidin-biotin affinity-based technology is discussed in greater detail above for whole cell capture. Such discussion applies equally to the methods of analysis of the present invention.

Techniques of analyzing useful in methods of the present invention involve standard techniques used in ELISA (e.g., colorimetric ELISA).

Preferably, in an assay of the present invention, the immobilized antibodies are physically adsorbed to a solid support material, such as a solid surface of, for example, a microwell plate. Preferably, a well of the microwell plate has immobilized therein a mixture of antibodies (e.g., a mixture of two monoclonal antibodies such as MAb 12-9 and MAb-76, or a mixture of a polyclonal antibody and an isolated monoclonal antibody).

Contacting the sample with the two or more immobilized antibodies can occur simultaneously and/or sequentially. Similarly, contacting the captured analytes, if present, with the two or more labeled antibodies can occur simultaneously and/or sequentially.

Contacting the sample with the immobilized antibodies under conditions effective to capture one or more analytes characteristic of a specific bacterium, if present in the sample, occurs under conditions to form one or more captured analytes. Preferably such conditions include conditions that have been optimized to assure effective antigen:antibody binding while minimizing nonspecific binding. Similarly, in contacting the one or more captured analytes, if present, with the labeled antibodies, conditions are used that are effective to cause binding between the one or more captured analytes and the labeled antibodies. Preferably such conditions include those that have been optimized to assure effective antigen:antibody binding while minimizing nonspecific binding.

Such conditions include the use of selective agents, such as salts, pH adjusting agents, surfactants, carrier proteins (e.g., albumen, casein, non-fat dry milk, serum, etc.), and the like, at levels that allow specific interactions of the binding pair (i.e., antigen, antibody) but minimizes other nonspecific (i.e., ionic and electrostatic) interactions.

Preferably, in an assay of the present invention, the labeled antibodies can be labeled with a wide variety of chemistries (often referred to as detectable or reporter moieties or markers) typically used for detection, which can include both direct and indirect methods of detection, which are well known in the art. In one method, a label can be a direct label (e.g., an enzyme such as horseradish peroxidase, glucose oxidase, alkaline phosphatase), which can be attached through a variety of attachment chemistries known in the art, such as that disclosed in P. Tijssen Practice and Theory of Enzyme Immunoassays, Laboratory Techniques in Biochemistry and Molecular Biology, Vol. 15, Elsevier Science Publishers, B.V. New York, N.Y. (1985).

Alternatively, a label can be an indirect label, and can be used in a system that includes, for example, biotin, which can be conjugated to the analyte-specific antibody thru a variety of chemistries know in the art. This indirect system, including a biotin-labeled antibody is then used with an enzyme conjugate (e.g., streptavidin- or avidin-bound enzymes such as streptavidin- or avidin-alkaline phosphatase conjugates), is well known in the art. Other indirect labels include, for example, an enzyme-labeled secondary antibody, haptens or other antigens that may be detected using labeled antibodies, as is known to one of skill in the art.

In certain embodiments, the antibodies are indirectly labeled with biotin, preferably biotin linked through a polyethylene oxide linker. If the antibody includes an indirect label such as this, the method can further include a step of reacting the labeled antibodies with an enzyme conjugate before, during, or after combining contacting the one or more captured analytes, if present, with the labeled antibodies.

In an exemplary system biotin can be linked to an antibody through a polyethylene oxide linker using N-hydroxysuccininimide chemistry, for example (e.g., PEO₄-NHS). In this embodiment, the biotin is considered the indirect label and the method includes reacting the labeled antibodies with an enzyme conjugate (including, for example, an enzyme linked to streptavidin or avidin). Such biotin-avidin system is a well known labeling system of the antibodies for detection, particularly when enzymatic detection systems are used as in an ELISA. The enzyme can be a wide variety of enzymes known for use in ELISA's, including, for example, alkaline phosphatase, horseradish peroxidase, glucose oxidase, etc. such as those disclosed in P. Tijssen Practice and Theory of Enzyme Immunoassays, Laboratory Techniques in Biochemistry and Molecular Biology, Vol. 15, Elsevier Science Publishers, B.V. New York, N.Y. (1985).

The enzyme can be detected colorimetrically using an appropriate chromogenic enzyme substrate, such as para-nitrophenyl phosphate for phosphatase enzyme labels, tetramethyl benzidine or diamino benzidine for peroxidase enzymes, and formazan salts as end products of glucose oxidation by glucose oxidase.

In addition to colorimetric detection, alternative methods of detection can include fluorimetric detection, detection of radiolabels, Raman spectroscopy, magnetic detection, luminescence, and eletrochemiluminescence, for example. Such methods of detection are well known to those of skill in the art.

The techniques used in the methods that utilize ELISA are generally well known to one of skill in the art. Significantly, however, the use of two or more immobilized antibodies having antigenic specificities for two or more distinct analytes characteristic of the specific bacterium, the use of a second set of two or more labeled antibodies having antigenic specificities for two or more distinct analytes characteristic of the specific microorganism, wherein, for each of the analytes present, the immobilized antibodies and the labeled antibodies comprise two or more antigen-binding pairs, provides significant advantage over conventional methods, particularly for analyzing bacteria such as S. aureus.

Significantly, using methods and devices of the present invention, improved sensitivity (i.e., lower levels of detection) and specificity can be realized relative to conventional detection methods for microorganisms, particularly bacteria such as S. aureus.

### III Acousto-Mechanical Methods of Analysis

The present invention also provides various acousto-mechanical-based methods of analyzing a sample for a bacterium of interest based on analysis of one or more analytes characteristic of the bacterium of interest. As with the enzyme-based methods, such methods can involve not only detecting the presence of one or more analytes characteristic of the bacterium of interest, but preferably identifying such analyte(s), which can lead to identifying a bacterium for which the analyte(s) is characteristic. In certain embodiments, analyzing the sample includes quantifying the total amount of analytes characteristic of the bacterium of interest.

Preferably, acousto-mechanical-based methods of the present invention include the use of two or more immobilized antibodies having antigenic specificities for two or more distinct analytes characteristic of the specific bacterium, the use of a second set of two or more antibodies in the form of particle-antibody conjugates, wherein the two or more antibodies have antigenic specificities for two or more distinct analytes characteristic of the specific microorganism, wherein, for each of the analytes present, the immobilized antibodies and the antibodies of the particle-antibody conjugates comprise two or more antigen-binding pairs. The antibodies of one set may be the same as the antibodies of the other set as long as they are capable of forming antigen-binding pairs.

Such antibodies are preferably cooperative in their binding characteristics. That is, they are capable of simultaneously binding to distinct regions of the target analyte(s) or optimally are found to be of complementary binding whereby the binding of their distinct analytes by one antibody set is enhanced by the binding of one or more other antibody sets.

Acousto-mechanical-based methods of the present invention include: providing a sample suspected of including one or more analytes characteristic of a specific bacterium; providing two or more particle—antibody conjugates having antigenic specificities for two or more distinct analytes characteristic of the specific bacterium; providing a system comprising an acousto-mechanical sensor that includes a detection surface comprising two or more immobilized antibodies having antigenic specificities for two or more distinct analytes characteristic of the specific bacterium; providing contact between the sample, the immobilized antibodies on the detection surface of the acousto-mechanical sensor, and the particle-antibody conjugates to bind the one or more analytes between the particle-antibody conjugates and the immobilized antibodies; wherein, for each of the analytes present, the immobilized antibodies and the particle-antibody conjugates comprise two or more antigen-binding pairs; and analyzing for the presence or absence of the specific bacterium.

As discussed above for the enzyme-based assays, the two or more distinct analytes can be separate molecules, such as protein A and Clumping Factor, or two different epitopes of the same molecule. The antibodies can include a solid phase label (i.e., particles) or the antibodies are the labels for the particles. Regardless of which is the detectable portion (i.e., label), such materials are referred to herein as particle-antibody conjugates.

Providing contact between the sample, the immobilized antibodies on the detection surface of the acousto-mechanical sensor, and the particle-antibody conjugates can occur simultaneously or sequentially in any order. For example, one method involves contacting the sample with the immobilized antibodies (i.e., the antibodies immobilized on the surface of the acousto-mechanical sensor) under conditions effective to capture one or more analytes characteristic of a specific bacterium, if present in the sample, to form one or more captured analytes; and contacting the one or more captured analytes, if present, with the particle-antibody conjugates under conditions effective to cause binding between the one or more captured analytes and the particle-antibody conjugates.

Alternatively, in certain embodiments of such methods, providing contact between the sample, the immobilized antibodies on the detection surface of the acousto-mechanical sensor, and the particle-antibody conjugates includes: contacting the sample with the particle-antibody conjugates, under conditions effective to cause interaction between the one or more analytes characteristic of the specific bacterium, if present in the sample, and the particle-antibody conjugates; and contacting the detection surface of the acousto-mechanical sensor with the sample containing the particle-antibody conjugates under conditions effective to cause binding between the particle-antibody conjugates, the one or more analytes, and the immobilized antibodies.

Conditions for binding the analytes between the immobilized antibodies and the particle-antibody conjugates can be determined readily by one of skill in the art.

In certain preferred embodiments, contacting the sample with the immobilized antibodies includes providing contact between the sample and each immobilized antibody simultaneously, although sequential contact can also be used. Analogously, in certain preferred embodiments, contacting the one or more captured analytes, if present, with the particle-antibody conjugates includes providing contact between the captured analytes and each particle-antibody conjugate simultaneously, although sequential contact can also be used, for example, if different the different antibodies are attached to different particles

Various methods can be used in obtaining binding between the particle-antibody conjugates, the one or more analytes, and the immobilized antibodies on the detection surface of the acousto-mechanical sensor. In one embodiment, particle-antibody conjugates with analyte attached thereto can be separated from the rest of the sample before contacting it with the detection surface of the acousto-mechanical sensor.

With the properly selected antibodies and an optimized system one could do any of the following: add the analyte to the acousto-mechanical sensor having immobilized antibodies (i.e., the sensor), then add the particle-antibody conjugates; add the analyte to the sensor, then add the antibody, then add the particles with specificity for “capping” the antibody; add the analyte to the particle-antibody conjugates, then add this to the sensor; add the analyte to the antibody, then add the particles to this mixture, and then add the mixture to the sensor; add the analyte to the antibody, add this to the sensor, and then add the particles to the sensor. Adding direct/indirect labeled antibodies to a sensor may be advisable to reduce background or to give a less reactive antibody (high Kd) a better chance to get to its target epitope. Another reason to do the sequential addition is to reduce nonspecific binding by adding more phase separations.

Typically, analyzing for the presence or absence of the specific bacterium can occur through the presence of one or more analytes or the absence of all analytes. If desired, the analysis can involve quantification of the total amount of captured analytes. This can occur using standard curves and methodology well known to one of skill in the art.

The present invention is advantageous over conventional techniques for analyzing samples for such bacteria because of the improved time required to obtain a useful result, ease of use, performance (sensitivity, specificity, etc.), and conservative (broad specie coverage) detection, etc.

In such acousto-mechanical-based methods, significantly, relatively small volumes of test sample can be used. Although test sample volume as high as 2 milliliters (mL) may be utilized, advantageously test samples on the order of 100 microliters (μL) are sufficient for methods of the present invention. As with ELISA, smaller test samples can be used, but less than 10-μL samples are not generally practical.

Using preferred acousto-mechanical-based methods of the present invention, the initial capture time can be relatively short. Also, the detection time can be relatively short. For example, the detection time can be less than 60 minutes, less than 30 minutes, less than 15 minutes, less than 10 minutes, and even as short as 5 minutes, depending on volume and flowrates.

Bacteria of particular interest include Gram positive bacteria and Gram negative bacteria as discussed above for whole cell capture and enzyme-based methods. Of particular interest are Gram positive bacteria, such as Staphylococcus aureus. Typically, these can be detected by detecting the presence of a cell-wall component characteristic of the bacteria, such as a cell-wall protein. Also, of particular interest are antibiotic resistant microbes including MRSA, VRSA, VISA, VRE, and MDR. Typically, these can be detected by additionally detecting the presence of an internal cell component, such as a membrane protein, transport protein, enzyme, etc., responsible for antibiotic resistance.

The acousto-mechanical-based methods of the present invention are advantageous over conventional techniques for analyzing samples for such microbes because the signal for the analyte (e.g., external cell-associated components and/or cell markers from the internal portion of the cells) characteristic of the bacterium is enhanced. Methods of the present invention could be used to analyze a sample for separate molecules (e.g., molecules like protein A and Clumping Factor for analysis of Staphylococcus aureus) or two different epitopes of the same molecule (e.g., a protein). Such analytes may be present on fragments of cells or whole cells, as discussed above.

Such analytes are the same as those discussed above for the enzyme-based methods (e.g., cell-wall proteins and internal cell components).

Species of interest can be analyzed in a test sample that may be derived from any of a wide variety of sources, such as a physiological fluid, as discussed above for whole cell capture and enzyme-based methods of analysis. Further, the test sample may be derived from a wide variety of body sites, as discussed above for whole cell capture and enzyme-based methods of analysis. Samples of particular interest include mucus-containing samples, particularly nasal samples, as discussed above for enzyme-based methods of analysis.

Besides physiological fluids, other test samples may include other liquids as well as solid(s) dissolved in a liquid medium, as discussed above for whole cell capture and enzyme-based methods of analysis. Samples can also include cultured cells.

Various patient sampling techniques, sample collection devices (e.g., swab), sample treatment procedures, etc., can be used as discussed above for whole cell capture and enzyme-based methods of analysis. Cells in a sample of interest can be either whole cells or lysed cells, as discussed above for enzyme-based methods of analysis. If the samples contain mucus (e.g., nasal samples), they can be treated with a mucolytic agent (and reagents as needed, e.g., surfactants) as discussed above for enzyme-based methods of analysis.

In the acousto-mechanical-based methods of the present invention, the sample is contacted with appropriate reactant molecules for analyte binding (e.g., an analyte-binding material that includes a bacterial-recognizing reagent). Such reactant molecules preferably include two or more antibodies. Such antibodies are attached to particulate material thereby forming particle-antibody conjugates, with the same or different antibodies that are attached to the detection surface of an acousto-mechanical sensor. The antibodies on the sensor surface are selected such that they allow non-competitive binding with the particulate-bound analyte complex.

Suitable antibodies are discussed above for whole cell capture and enzyme-based methods of analysis (e.g., including capture and labeling). These include, for example, monoclonal antibodies, polyclonal antibodies, or combinations thereof, fragments thereof, etc. Specific examples include those selected from the group consisting of MAb-76, MAb-107, affinity-purified RxClf40, affinity-purified GxClf40, MAb 12-9), fragments thereof, and combinations thereof. Preferred antibodies are monoclonal antibodies. Particularly preferred are monoclonal antibodies that bind to Protein A of Staphylococcus aureus (also referred to herein as “S. aureus” or “Staph A”). In certain preferred embodiments of the methods of analysis of the present invention, the two or more immobilized antibodies include two (isolated) monoclonal antibodies, particularly MAb12-9 and MAb-76. In certain preferred embodiments of the present invention, the two or more antibodies in the particle-antibody conjugates include one monoclonal antibody, such as MAb-107, and one polyclonal antibody, such as affinity-purified RxClf40.

Antibodies can be attached to particulate material and immobilized on the detection surface of an acousto-mechanical sensor using a variety of techniques (e.g., covalent or non-covalent), as discussed above for whole cell capture and enzyme-based assays.

The particles of the particle-antibody conjugates, as well as the surface of the acousto-mechanical sensor can have one or more antibodies disposed thereon. For example, one type of antibody can be disposed on one type of particulate material and another type of antibody on the same or different type of particulate material, but on different particles. In certain embodiments, the solid support material (e.g., each particle or the sensor detection surface) has at least two antibodies that bind different analytes disposed thereon. For example, in certain embodiments, the particle-antibody conjugates include particulate material has antibodies MAb-76 and MAb 12-9 disposed thereon, preferably disposed on each particle of the particulate material. Depending on the desired results and antibodies used, the concentrations and densities of immobilized antibodies can be varied by one of skill in the art.

Contacting the analytes, if present, with the two or more antibodies immobilized antibodies can occur simultaneously and/or sequentially. Similarly, contacting the captured analytes, if present, with the two or more particle-antibody conjugates can occur simultaneously and/or sequentially. Conditions are used that are effective to cause binding between the analytes, the immobilized antibodies on the sensor detection surface, and the particle-antibody conjugates. Preferably such conditions include those that have been optimized to assure effective antigen:antibody binding while minimizing nonspecific binding. Such conditions include the use of selective agents, such as salts, pH adjusting agents, surfactants, carrier proteins (e.g., albumen, casein, non-fat dry milk, serum, etc.), and the like, at levels that allow specific interactions of the binding pair (i.e., antigen, antibody) but minimizes other nonspecific (i.e., ionic and electrostatic) interactions.

The techniques used in the methods that utilize acousto-mechanical sensors are generally well known to one of skill in the art. Significantly, however, the use of two or more immobilized antibodies having antigenic specificities for two or more distinct analytes characteristic of the specific bacterium, the use of a second set of two or more antibodies in particle-antibody conjugates, wherein the antibodies have antigenic specificities for two or more distinct analytes characteristic of the specific microorganism, wherein, for each of the analytes present, the immobilized antibodies and the particle-antibody conjugates comprise two or more antigen-binding pairs, provides significant advantage over conventional methods, particularly for analyzing bacteria such as S. aureus.

Significantly, using methods and devices of the present invention, improved sensitivity (i.e., lower levels of detection) and specificity can be realized relative to conventional detection methods for microorganisms, particularly bacteria such as S. aureus.

In acousto-mechanical-based methods of the present invention, the presence of target biological analyte in a test sample is detected through the use of acousto-mechanical energy that is measured or otherwise sensed to determine wave attenuation, phase changes, frequency changes, and/or resonant frequency changes. The acousto-mechanical energy may be generated using, e.g., piezoelectric-based surface acoustic wave (SAW) devices. As described in, e.g., U.S. Pat. No. 5,814,525 (Renschler et al.), the class of piezoelectric-based acoustic mechanical devices can be further subdivided into surface acoustic wave (SAW), acoustic plate mode (APM), or quartz crystal microbalance (QCM) devices depending on their mode of detection. Various acousto-mechanical sensors are known, and are described in PCT App. Ser. No. US 2007/075948, filed Aug. 15, 2007, as well as other patents and publications referenced below.

In particular embodiments, the acousto-mechanical-based methods of the present invention involve capturing the target analytes on the surface of magnetic particles and then capturing the magnetic particles on a sensor surface. The use of magnetic particles increases the amount of target analyte that can be captured on the sensor surface using magnetophoresis. Further, the coupling of the magnetic particles (via the target analyte) to the sensor surface can enhance the sensor response. Capturing target biological analytes with a coated magnetic particle is disclosed in PCT App. Ser. No. US 2007/075948, filed Aug. 15, 2007.

The capture of the magnetic particles attached to the target analytes on the sensor surface is influenced by several factors including the strength of the magnetic field (as determined by the strength of the magnet and the distance of the magnet from the sensor), the location of the magnetic field generator relative to the sensor surface in the X-Y plane, the orientation of the magnetic field generator, the size and composition of the magnetic particle, and the flow rate of the test sample over the sensor during capture and movement of the magnetic field generator. For instance, one may place the magnetic field generator immediately adjacent the sensor surface (for example, at a zero mm distance) and capture all particles at the leading edge of the sensor for a given flow rate. The magnetic field generator then may be removed to release the cluster of particles to flow over the sensor. Alternately, the magnetic field generator may be positioned some distance away from the sensor while still maintaining a magnetic field that attracts the magnetic particles, and with the use of an appropriate flow rate the particles may be uniformly coated over the sensor in a single step.

After all the magnetic particles in the sensor have been captured on the sensor surface, the magnetic field generator is removed in order for the sensor to provide a signal that correlates to the analyte concentration. In one embodiment utilizing the methods described herein, the sensor can detect as low as 1 ng per 500 microliters of target biological analyte in the sample material.

Additionally, the magnetic particles used are preferably less than one micron in size when used with a SH-SAW sensor. In a preferred embodiment, the magnetic particles are 250 microns, and more preferably 100 microns in size. The use of magnetic particles to bind to the target analyte provides an enhancement in capture efficiency of target analytes on the sensor surface. Significantly higher amounts of target analyte can be captured on the sensor surface. For example, relying on diffusion alone leads to a capture of approximately 0.1 to 1% of the available target analytes passing over the sensor. However, with the application of the magnetic field generator to the magnetic particles bound to target analytes, a capture efficiency of up to 100% of the target analyte on the sensor surface is possible using magnetophoresis. Because the target analytes are bound on the magnetic particles drawn to the sensor surface, the target analytes are moved to the surface at much higher rates than other constituents of the sample. Hence preferential attachment of the target analyte to the sensor surface is achieved.

The methods described herein employ an acoustic sensor, and more specifically, an acoustic mechanical biosensor, that detects a change in at least one physical property and produces a signal in response to the detectable change. Preferably, the acoustic mechanical biosensor employed herein is a surface acoustic wave (SAW) biosensor. In these devices an acoustic wave is generated from an interdigitated transducer (IDT) on a piezoelectric substrate either as a surface acoustic wave or as a bulk acoustic wave. A second IDT may convert the acoustic wave back to an electric signal for measurement. This is referred to as a delay line. Alternatively the device may operate as a resonator. The space between the two IDTs can be modified with a coating that may include reactive molecules for chemical or biosensing applications.

Piezoelectric-based SAW biosensors typically operate on the basis of their ability to detect minute changes in mass or viscosity. As described in U.S. Pat. No. 5,814,525, the class of piezoelectric-based acoustic mechanical biosensors can be further subdivided into surface acoustic wave (SAW), acoustic plate mode (APM), or quartz crystal microbalance (QCM) devices depending on their mode of detection of mass changes.

In some embodiments, the acoustic mechanical biosensor includes a secondary capture agent or reactant (e.g., antibody) that attaches the target analyte to the surface of the piezoelectric acoustic mechanical biosensor. The propagation velocity of the surface wave is a sensitive probe capable of detecting changes such as mass, elasticity, viscoelasticity, conductivity and dielectric constant. Thus, changes in any of these properties results in a detectable change in the surface acoustic wave. That is, when a substance comes in contacts with, absorbs, or is otherwise caused to adhere to the surface coating of a SAW device, a corresponding response is produced.

APM can also be operated with the device in contact with a liquid. Similarly, an alternating voltage applied to the two opposite electrodes on a QCM (typically AT-cut quartz) device induces a thickness shear wave mode whose resonance frequency changes in proportion to mass changes in a coating material.

The direction of the acoustic wave propagation (e.g., in the plane parallel to the waveguide or perpendicular to the plane of the waveguide) is determined by the crystal-cut of the piezoelectric material from which the acoustic mechanical biosensor is constructed. SAW biosensors that have the majority of the acoustic wave propagating in and out of the plane (i.e., Rayleigh wave, most Lamb-waves) are typically not employed in liquid sensing applications since there is too much acoustic damping from the liquid contact with the surface.

For liquid sample mediums, a shear horizontal surface acoustic wave biosensor (SH-SAW) is preferably used, and is constructed from a piezoelectric material with a crystal-cut and orientation that allows the wave propagation to be rotated to a shear horizontal mode, i.e., in plane of the biosensor waveguide), resulting in reduced acoustic damping loss to the liquid in contact with the biosensor surface. Shear horizontal acoustic waves include, e.g., acoustic plate modes (APM), surface skimming bulk waves (SSBW), Love-waves, leaky acoustic waves (LSAW), and Bleustein-Gulyaev (BG) waves.

In particular, Love mode sensors consist of a substrate supporting a SH wave mode such as SSBW of ST quartz or the leaky wave of 36° YXLiTaO₃. These modes are converted into a Love-wave mode by application of thin acoustic guiding layer or waveguide. These waves are frequency dependent and can be generated provided that the shear wave velocity of the waveguide layer is lower than that of the piezoelectric substrate. SiO₂ has been used as an acoustic waveguide layer on quartz. Other thermoplastic and crosslinked polymeric waveguide materials such as polymethylmethacrylate, phenol-formaldehyde resin (e.g., trade designation NOVALAC), polyimide and polystyrene, have also been employed.

Alternatively QCM devices can also be used with liquid sample mediums, although with these devices the acoustic wave will be severely damped by the liquid medium, leading to a generally less sensitive device.

Biosensors employing acoustic mechanical means and components of such biosensors are known. See, for example, U.S. Pat. Nos. 5,076,094; 5,117,146; 5,235,235; 5,151,110; 5,763,283; 5,814,525; 5,836,203; 6,232,139. SH-SAW devices can be obtained from various manufacturers such as Sandia National Laboratories, Albuquerque, N. Mex. Certain SH-SAW biosensors are also described in “Low-level detection of a Bacillus anthracis stimulant using Love-wave biosensors of 36° YXLiTaO₃,” Biosensors and Bioelectronics, 19, 849-859 (2004). SAW biosensors, as well as methods of detecting biological agents, are also described in U.S. Pat. App. Ser. No. 60/533,169, filed Dec. 30, 2003.

In some embodiments, the surface of the biosensor includes a secondary capture agent or reactant (e.g., antibody) overlying the waveguide layer. In this embodiment, the biosensor typically detects a change in viscosity and/or mass bound by the secondary capture agent or reactant. For this embodiment, the biosensor preferably includes an immobilization layer (overlying the waveguide layer) and optional tie layer(s).

Examples of techniques for driving and monitoring acousto-mechanical sensors such as those that may be used in connection with the present invention may be found in, e.g., U.S. Pat. Nos. 5,076,094 (Frye et al.); 5,117,146 (Martin et al.); 5,235,235 (Martin et al.); 5,151,110 (Bein et al.); 5,763,283 (Cernosek et al.); 5,814,525 (Renschler et al.); 5,836,203 ((Martin et al.); and 6,232,139 (Casalnuovo et al.), etc. Further examples may be described in, e.g., Branch et al., “Low-level detection of a Bacillus anthracis simulant using Love-wave biosensors on 36° YX LiTaO₃,” Biosensors and Bioelectronics, 19, 849-859 (2004); as well as in PCT Pub. No. WO 2005/066622, filed on Dec. 17, 2004.

A detection apparatus may be contained in an integrated unit that may be described as a detection cartridge. Exemplary detection cartridges are further described in PCT Pub. No. WO2005/075973, filed Dec. 17, 2004 and PCT Publication No. WO2005/064349, filed on Dec. 17, 2004, which describe additional features of detection cartridges and/or modules that may be used in connection with the present invention. Other optional features of the sensor cartridge, such as fluid monitors and modules for delivering various materials are further described in PCT Pub. No. WO2005/075973. Additional discussion related to various detection systems and components (such as detection cartridges including biosensors) may be found in, e.g., PCT Pub. Nos. WO2005/075973 and WO2005/064349.

The methods of the present invention may be utilized in combination with various materials, methods, systems, apparatus, etc. as described in various U.S. patent applications identified below. They include U.S. Pat. App. Pub. Nos. 2005-0142296-A1; 2005-0107615-A1; 2005-0112672-A1; 2005-0106709-A1; 2005-0227076-A1; U.S. Pat. App. Pub. Nos. 2005-0153370-A1; 2006-0135718-A1; 2006-0135783-A1; PCT Pub. No. WO 2005/066622; PCT Pub. No. WO 2005/066621; PCT Publication No. WO2005/075973; PCT Pub. No. WO2005/064349; and PCT Pub. No. WO2005/066092.

### EXAMPLES

The present invention has now been described with reference to several specific embodiments foreseen by the inventor for which enabling descriptions are available. Insubstantial modifications of the invention, including modifications not presently foreseen, may nonetheless constitute equivalents thereto. Thus, the scope of the present invention should not be limited by the details and structures described herein, but rather solely by the following claims, and equivalents thereto.

### I. Methods of Capturing Bacterial Whole Cells

### Example I-1

### Preparation of Individual Antibody-Magnetic Particle Conjugates

### A. One-Micron Particles.

Murine anti-Protein A monoclonal antibody, MAb-107, is described in U.S. patent application Ser. No. 11/562,747, filed on Nov. 22, 2006, and PCT App. Ser. No. U.S. Ser. No. 07/84,739, both entitled “ANTIBODY WITH PROTEIN A SELECTIVITY.” Murine anti-Staphylococcal clumping factor monoclonal antibody, MAb12-9, described in U.S. Pat. No. 6,979,446, was obtained from Inhibitex, Inc. (Alpharetta, Ga.). Affinity purified goat anti-clumping factor immune serum is described in U.S. Pat. App. Ser. No. 60/867,089, filed on Nov. 22, 2006, and U.S. Pat. App. Ser. No. ______ (Attorney Docket No. 62611US005), filed on even date herewith, both of which are entitled “SPECIFIC ANTIBODY SELECTION BY SELECTIVE ELUTION CONDITIONS.”

All antibody preparations were biotinylated with EZ-Link NHS-PEO4-Biotin (Product Number 21330) from Pierce according to the manufacturer's directions. Streptavidin-coated magnetic particles (1-μm Dynal T1) were obtained from Invitrogen, Inc. (Carlsbad, Calif.). All reactions and washes were performed in PBS L-64 buffer (phosphate buffered saline with 0.2% w/v PLURONIC L64) unless stated otherwise. Wash steps included three successive washes unless stated otherwise. The washing process consisted of placing a magnet adjacent to the tube to draw the particles to the side of the tube proximal to the magnet, removing the liquid from the tube with the adjacent magnet, and adding an equal volume of fresh buffer to replace the liquid that was removed. The magnet was removed to allow resuspension and mixing the particles.

Streptavidin-coated magnetic particles, at a concentration of 2.5 milligrams per milliliter (mg/mL) were mixed with biotinylated antibody preparations in 500 microliter (μL) PBS L-64 buffer. The mass ratio of the antibody to the particles for conjugation was 40 μg antibody/mg of particles. The resulting mixture was incubated at 37° C. for 1 hour (hr). Subsequently, the particles were washed in PBS L-64 buffer to remove unbound antibody. After the final wash the particles were resuspended to a particle concentration of 2.5 mg/mL.

### B. Nanoparticles.

Streptavidin coated 200-nm particles were obtained from Ademtech (Pessac, France). The biotinylated MAb-107 antibody was conjugated to the streptavidin-coated particles according to the method described above with the exception that the antibody binding reaction was carried out in the proprietary immobilization buffer provided by the manufacturer (Ademtech). The antibody to particles mass ratio for conjugation was 1 μg antibody/mg of particles for the Ademtech particles. After washing the particles, the antibody-conjugated particles were resuspended in immobilization buffer at a particle concentration of 5 mg/mL.

### Example I-2

### Preparation of Magnetic Particle Suspensions Containing More than One Antigen Specificity

### A. Preparation of Immunopolyspecific Magnetic Particles.

A suspension of streptavidin-coated Dynal T1 magnetic particles (2.5 mg/mL) was conjugated with a mixture of affinity-purified GxClf40-biotin and MAb107-biotin as described in Example I-1A.

In this example, the mass ratio of each individual antibody to the particles for conjugation was 20 mg antibody/mg particles. After washing the particles, they were resuspended in PBS L-64 to a final particle concentration of 2.5 mg/mL. This suspension was called “Mixture 1”.

A suspension of streptavidin-coated Ademtech 200-nm magnetic particles (2.5 mg/mL) was conjugated with a mixture of MAb107-biotin and affinity-purified GxClf40-biotin antibodies as described in Example I-1B except that the mass ratio of each antibody to the particles in the conjugation reaction was adjusted to 1.0 μg antibody/mg particles in the immobilization buffer. After washing, the particles were resuspended in immobilization buffer at a particle concentration of 5 mg/mL. This suspension was called “Mixture 2”.

### B. Preparation of Mixtures of Monospecific Antibody-Coated Magnetic Particles.

Equal volumes of the MAb107 and affinity-purified GxClf40 antibody-conjugated Dynal T1 (1-μm) particle suspensions from Example I-1A were mixed to provide a final particle concentration of 2.5 mg/mL (1.25 mg/mL of the MAb-107 conjugate and 1.25 mg/mL of the affinity-purified GxClf40 conjugate). The resulting suspension was called “Mixture 3”.

### Example I-3

### Capture of Staphylococcus aureus Cells Using Antibody-Coated 1-μm Magnetic Particles

### A. Methods.

S. aureus strains used for the cell capture studies are listed in Table I-1. Strain 25923 was obtained from the American Type Culture Collection (Manassas, Va.). The other strains of S. aureus were isolated from clinical specimens. Bacterial suspensions were prepared using overnight cultures grown in tryptic soy broth at 37° C. The cultures were centrifuged to harvest the cells and the cell pellets were resuspended in sterile phosphate buffered saline with 0.2% w/v PLURONIC L64 to a final concentration of approximately 5×10⁸ cfu/mL. Prior to experimentation, the bacteria were washed in triplicate and diluted to approximately 5×10³. Thirty-two microliters of antibody-coated (Dynal) particle suspensions (2.5 mg/mL) were mixed with 468 microliters of the S. aureus suspension in a polypropylene 2-mL vial. The vial was manually rocked for 30 seconds (s) to mix the bacteria and the particles and then agitated on a rocking platform (Reciprocating Barnstead/Thermolyne Varimix set at approximately 0.3 cycle per second) for 15 minutes. The particles were drawn to one side of the vial (with a magnet, as in Example I-1) for 5 minutes. The supernatant, containing bacterial cells that were not adsorbed by the particles, was removed and diluted 10-fold in sterile PBS L-64. The vial was removed from the magnet, 500 μL, sterile PBS L-64 buffer was added, and the vial was manually agitated for 30 seconds (s) to resuspend the particles. The vial was placed adjacent to the magnet for 5 minutes and the wash solution was aspirated and diluted 10-fold in PBS L-64. The vial was removed from the magnet, 500 μL of buffer was added, and the vial was manually agitated for 30s to resuspend the particles.

The number of viable bacteria in each of the respective solutions (supernatant, wash, and resuspended particles) was determined by plating serial dilutions of each suspension on PETRIFILM Aerobic Count Plates (3M Company, St. Paul, Minn.).

TABLE I-1
Bacterial strains.

| Strain Number | Source |
|---|---|
| 16 | Clinical specimen |
| 207 | Clinical specimen |
| 221 | Clinical specimen |
| 271 | Clinical specimen |
| 317 | Clinical specimen |
| 329 | Clinical specimen |
| 336 | Clinical specimen |
| 25923 | American Type Culture Collection |

B. S. aureus Capture with Individual Antibody-Conjugated Magnetic Particles.

Conjugates of Dynal T1 particles (Example I-1A) were used to capture and quantitate staphylococcal cells according to Example I-3A. Strain 25923 produces both Protein A and clumping factor antigens. Strain 317 produces clumping factor, but does not produce detectable levels of Protein A, using an ELISA assay with lysed staphylococcal cells as the test antigen. Replicate samples were run for each antibody. The results are shown in Table I-2.

TABLE I-2
Capture of S. aureus strains using antibody-conjugated particles.

| | % S. aureus Captured | | | |
|---|---|---|---|---|
| | Strain 25923 | Strain 25923 | Strain 317 | Strain 317 |
| Antibody | rep 1 | rep 2 | rep 1 | rep 2 |
| Mab 107 | 70.6 | 75.2 | 2.8 | 2.4 |
| GxClfa | 76.4 | 81.2 | 82.8 | 84.2 |
| Mab 12-9 | 74 | 77.9 | 72.5 | 72.2 |

The data indicate that, although both strains were efficiently captured by particles coated with either of the anti-clumping factor antibodies, strain 317 was not efficiently captured by the particles coated with anti-Protein A antibodies.

C. Capture of S. aureus Strain 317 Using Particle Suspensions Containing Antibodies Directed Against Both Protein a and Clumping Factor Antigens.

Conjugates of Dynal T1 particles with Mab107 and affinity-purified GxClf40 antibodies, respectively, were prepared as described in Example I-2A (Mixture 1). Conjugates of Dynal T1 particles with both Mab107 and affinity-purified GxClf40 antibodies were prepared according to Example I-2B (Mixture 3). Suspensions of S. aureus strain 317 were produced, contacted with the magnetic particles suspensions, and bacterial capture was measured as described in Example I-3A. The results are summarized in Table I-3.

TABLE I-3
S. aureus Strain 317 capture with Mab107, affinity-purified
GxClf40 antibodies and their mixtures.

% S. aureus Captured

| Antibody | rep 1 | rep 2 |
|---|---|---|
| Mab 107 | 1.0 | 0.8 |
| GxClfa | 82.9 | 80.6 |
| Mixture 1 | 80.2 | 79.5 |
| Mixture 3 | 56.1 | 57.1 |

As observed in Example I-3B, there was very little capture of the cells by MAb-07 antibody-conjugated particles and efficient capture of cells by affinity-purified GxClf40 antibody-conjugated particles. The data show efficient capture of S. aureus cells by particle suspensions that have both antibody specificities, whether the antibodies were conjugated to the particles together, or they were conjugated to the particles separately.

Similar capture experiments were repeated with several other strains of S. aureus and the results are shown in Table I-4.

TABLE I-4
S. aureus capture with monospecific anti-Protein A (MAb-107)
and polyspecific anti Protein A and anti-clumping factor
(MAb-107 + affinity-purified. GxClf40) particles.

| Strain | Mab 107 | Mixture 1 |
|---|---|---|
| 317 | 0.9 | 79.8 |
| 207 | 1.4 | 21.9 |
| 221 | 23.0 | 69.5 |
| 336 | 62.9 | 60.8 |

The particles coated with both antibodies showed significantly better capture of the three strains that express little or no Protein A. For the fourth strain (336), which expresses relatively high levels of Protein A, the cell capture was comparable for both monospecific and polyspecific particles.

### Example I-4

### Capture of Staphylococcus aureus Cells Using Antibody-Coated 200-nm Magnetic Particles

A. Capture with Immunomonospecific (Anti-Protein A) Particles.

Bacterial suspensions were grown, harvested, and washed as described in Example I-3. Ademtech 200-nm magnetic particles were coated with MAb-107 antibody as described in Example I-1B. Bacterial capture experiments were performed as in Example I-3 except that, in this example, 15.8 microliters of antibody-conjugated particles were mixed with 484 microliters of bacterial suspension. Five different bacterial strains were used in this example. The capture results are shown in Table I-5.

B. Capture with Immunopolyspecific (Anti-Protein A+Anti-Clumping Factor) Particles.

Bacterial suspensions were grown, harvested, and washed as described in Example I-3. Ademtech 200-nm magnetic particles were coated with MAb-107 and affinity-purified GxClf40 antibodies as described in Example I-2A. Bacterial capture experiments were performed as in Example I-3 except that, in this example, 15.8 microliters of antibody-conjugated particles were mixed with 484 microliters of bacterial suspension. Five different bacterial strains were used in this example. The capture results are shown in Table I-5.

TABLE I-5
MAb-07 antibody vs. MAb-107 + affinity-purified
GxClf40 dual antibody system for different strains of
S. aureus using Ademtech 200-nm particles.

| Strain | Mab 107 | Mixture 2 |
|---|---|---|
| 16 | 75.2 | 77.9 |
| 207 | 0.9 | 22.8 |
| 221 | 18.4 | 54.0 |
| 271 | 41.8 | 53.0 |
| 329 | 94.7 | 94.9 |

As observed with the larger (1-μm) magnetic particles, these results demonstrate that particles coated with a mixture of antibodies having different antigenic specificities more efficiently captured the S. aureus cells that express relatively low levels of Protein A.

### II. Methods of Analyzing Samples for Bacteria Using ELISA

### Example II-1

### Optimization of Concentrations of Capture and Capping Antibodies Used in the Tandem Protein A/Clumping Factor Enzyme Linked Immunosorbant Assay (ELISA)

### A. General Materials and Methods.

The antibodies used in the assay included two anti-Staphylococcus aureus protein A monoclonal antibodies (MAb-76 and MAb-107, which are disclosed in U.S. Pat. App. Ser. No. 11/562,759, filed on Nov. 22, 2006, and PCT App. Ser. No. U.S. Ser. No. 07/84,736, both entitled “ANTIBODY WITH PROTEIN A SELECTIVITY,” or in U.S. patent application Ser. No. 11/562,747, filed on Nov. 22, 2006, and PCT App. Ser. No. U.S. Ser. No. 07/84,739, both entitled “ANTIBODY WITH PROTEIN A SELECTIVITY”), the anti-Staphylococcus aureus clumping factor monoclonal antibody, MAb 12-9 (Inhibitex, Alpharetta, Ga., described in U.S. Pat. No. 6,979,466), and RxClf40 (rabbit anti-Clf40 antisera, Inhibitex). Prior to use in the ELISA assays, the RxClf40 antibody was affinity purified according to the process described in U.S. Pat. App. Ser. No. 60/867,089, filed on Nov. 22, 2006, and U.S. patent application Ser. No. ______ (Attorney Docket No. 62611US005), filed on even date herewith, both of which are entitled “SPECIFIC ANTIBODY SELECTION BY SELECTIVE ELUTION CONDITIONS.”

All capping antibodies were biotinylated prior to use. Antibodies were biotinylated according to the manufacturer's instructions using the EZ-Link NHS-PEO4-Biotin kit from Pierce (Rockford, Ill.).

Phosphate buffered saline (PBS, 137 mM NaCl and 2.7 mM KCl in 10 mM phosphate buffer, pH 7.50) used in these experiments was prepared from a 10× concentrated solution obtained from EMD Biosciences (San Diego, Calif.). The PBST reagent was prepared by adding 0.05% (v/v) TWEEN 20 to the PBS buffer. Costar 96-well high-binding polystyrene microtiter plates were obtained from Corning LifeSciences (Acton, Mass.). All buffers were filtered prior to use except the wash buffer. All procedures were performed at room temperature unless specified otherwise. All ELISA wash procedures included five sequential wash volumes of 200 microliters per well and all washes were done with PBST buffer. Alkaline phosphatase chromogenic substrate, pNPP, was obtained from KPL (Gaithersburg, Md.).

Antigens used in the optimization experiments included Protein A antigen from Zymed Laboratories (Invitrogen, Inc. Carlsbad, Calif.), clumping factor antigen (rClf40, U.S. Pat. No. 6,008,341) from Inhibitex (Alpharetta, Ga.) and lysed cells of S. aureus strain 25923. S. aureus 25923 was obtained from the American Type Culture Collection (Manassas, Va.). All antigens were prepared in filtered lysing solution lysostaphin (Sigma Aldrich, St. Louis, Mo.) diluted to 3 μg/mL in antigen dilution buffer. Antigen dilution buffer consisted of PBS containing 0.2% w/v PLURONIC L64 (BASF, Florham Park, N.J.) and 50 mM disodium EDTA, pH 7.44.

To prepare the S. aureus antigen, strain 25923 was grown at 37° C. in tryptic soy broth (Hardy Diagnostics, Santa Maria, Calif.) overnight to an estimated concentration of 5.0×10⁸ cfu/mL. The culture was washed twice (centrifuged at 4° C. for 10 minutes at 10,000 rpm), then resuspended in PBSL (PBS containing 0.2% w/v PLURONIC L64, BASF, Florham Park, N.J.). The washed bacterial stock was diluted 1:50 in PBSL to an approximate concentration of 1.0×10⁷ cfu/mL. This was further diluted 1:100 into lysing solution, for a final sample concentration of 1.0×10⁵ cfu/mL. Unlysed, diluted bacteria were plated on blood agar plates to verify the concentration of the original broth cultures. Assay results reflect the actual starting bacterial concentration.

### B. Elisa Assay Conditions.

One or two capture antibodies were used in the assay. The antibodies were diluted from their refrigerated (4° C.) storage starting concentrations to microwell coating concentrations in PBS. One hundred microliters of the coating solutions were added to the wells of the microwell plates. Plates were incubated at 37° C. for 60 minutes. The coating solutions were removed prior to the blocking step.

All microtiter plates were blocked with 200 μL/well of StabilCoat immunoassay stabilizer (Surmodics, Eden Prairie, Minn.), then incubated overnight at 4° C. The blocking solution was removed prior to the antigen coating step.

One-hundred microliters of the appropriate solution of capture antigens were added to each well and the plates were incubated for 60 minutes at 37° C. The plates were washed subsequently with PBST solution.

Two capping antibodies were used in the assay. All capping antibody preparations were diluted in blotto (2% dried nonfat milk in PBST). One hundred microliters of the appropriate capping antibody mixture was added to each well and the plates were incubated for 60 minutes at 37° C.

Streptavidin-conjugated alkaline phosphatase (Streptavidin AP, Jackson ImmunoResearch Laboratories, Inc., West Grove, Pa.) was diluted in blotto to a concentration of 0.5 μg/mL. The microtiter plates were washed, and 100 μL/well of the Streptavidin AP was added to the plates. The plates were incubated for 60 minutes at 37° C.

Plates were washed. Subsequently, 100 μL/well of pNPP substrate was added to the plates. The plates were held at room temperature for 15 minutes to observe the development of color. The alkaline phosphatase reaction was stopped by adding 100 μL/well of 5% (w/v) disodium EDTA and the plates were placed in a plate reader, where the absorbance at 405-nm wavelength was read.

### C. Optimization of Capture Antibody Concentrations and Ratios for the Detection of Staphylococcal Antigens.

ELISA assays were set up as described above. The objective of this study was to determine which concentrations of capture antibodies and capping antibodies provide the best binding (highest absorbance reading in the ELISA assay) with all three antigen preparations and, concommitantly, provide the lowest background (lowest absorbance reading in the ELISA assay). Seven 96-well plates were coated with the capture antibody combinations shown in Table II-1.

TABLE II-1
Capture antibody concentrations used in optimization study.

| | | MAb 12-9 |
|---|---|---|
| Column | MAb-76 | anti-clumping |
| Number | anti-protein A | factor |
| 1 | 7.5 | 0 |
| 2 | 7.5 | 7.5 |
| 3 | 3.75 | 7.5 |
| 4 | 1.88 | 7.5 |
| 5 | 0.93 | 7.5 |
| 6 | 0.47 | 7.5 |
| 7 | 0 | 7.5 |
| 8 | 3.5 | 3.5 |
| 9 | 7.5 | 3.75 |
| 10 | 7.5 | 1.88 |
| 11 | 7.5 | 0.93 |
| 12 | 7.5 | 0.47 |

All concentrations are listed in μg/mL.

Four antigens were run in the assay: 1) PBS (no antigen control); 2) rClf40 clumping factor protein diluted in lyse solution to 1 ng/mL; 3) Zymed Protein A diluted in lyse solution to 100 pg/mL; and 4) S. aureus strain 25923 (ATCC, Manassas, Va.), prepared as described above. One hundred microliter aliquots of antigen were added to the plates and incubated as described above.

Seven tandem capping antibody mixtures were made, one mixture for each microtiter plate. The capping antibody mixtures are shown in Table II-2.

TABLE II-2
Capping antibody concentrations used in optimization study.

| | | Rabbit anti- |
|---|---|---|
| Plate | MAb-107.biotin | clumping |
| Number | anti-protein A | factor.biotin |
| 1 | 2.5 | 0 |
| 2 | 0 | 2.5 |
| 3 | 2.5 | 2.5 |
| 4 | 1.25 | 2.5 |
| 5 | 2.5 | 1.25 |
| 6 | 2.5 | 0.625 |
| 7 | 0.625 | 2.5 |

All concentrations are listed in μg/mL.

Table II-3 shows representative ELISA results from one of the plates that were used in the study. The data from all of the experiments indicated that the combination of capture antibodies concentrations consisting of 0.93 μg/mL MAb-76 and 7.5 μg/mL MAb 12-9 gave the highest binding activities for the antigens and the lowest background readings for the PBS controls. This combination was selected for further optimization of the capping antibody mixture.

TABLE II-3
ELISA assay results for the detection of Staphylococcal Clumping
factor, Protein A, and lysed Staphyloccus aureus antigens.
Capping Antibodies = 2.5 μg/mL RxClf40 + 2.5 μg/mL MAb-107

| | [MAb-76] | | | | | | | | | | | |
|---|---|---|---|---|---|---|---|---|---|---|---|---|
| | 7.5 | 7.5 | 3.75 | 1.88 | 0.93 | 0.47 | 0 | 3.5 | 7.5 | 7.5 | 7.5 | 7.5 |
| [MAb 12-9] | 0 | 7.5 | 7.5 | 7.5 | 7.5 | 7.5 | 7.5 | 3.5 | 3.75 | 1.88 | 0.93 | 0.47 |
| 0 | 0.344 | 0.261 | 0.179 | 0.144 | 0.120 | 0.108 | 0.095 | 0.178 | 0.277 | 0.279 | 0.310 | 0.281 |
| ClfA | 0.421 | 2.566 | 2.416 | 2.268 | 2.197 | 2.164 | 2.203 | 2.806 | 2.240 | 1.752 | 1.323 | 0.944 |
| ProtA | 1.032 | 0.917 | 0.815 | 0.701 | 0.634 | 0.582 | 0.116 | 0.825 | 0.886 | 0.896 | 0.939 | 0.930 |
| 25923 | 3.905 | 3.887 | 3.908 | 3.723 | 3.603 | 3.233 | 0.232 | 3.852 | 3.844 | 3.819 | 3.894 | 3.859 |

The capping antibodies in this study were biotin labeled affinity purified rabbit anti-clumping factor and biotin labeled MAb-107, both at a concentration of 2.5 μg/mL. The concentrations of each capture antibody (MAb-76 and Mab 12-9, respectively) in the various combinations are shown in μg/mL. The data are the average absorbance readings at 405 nm for a minimum of two duplicate wells. This table is representative data from a number of experiments that were done. Each experiment involved the use of different concentrations of capping antibodies.

### D. Optimization of Capping Antibody Concentrations for the Detection of Staphylococcal Antigens.

This experiment was conducted similar to the one described in Example II-1C except that the capture antibody concentrations were held constant at 1 μg/mL (MAb-76) and 7.5 μg/mL (MAb 12-9). Representative data from these experiments are shown in Table II-4. The data indicate that a mixture of capping antibodies consisting of MAb-107 at 2.5 μg/mL and affinity-purified RxClf40 at 0.75 μg/mL resulted in the detection of the lowest amounts of staphylococcal antigens.

TABLE II-4
ELISA assay results for the detection of Staphylococcal Clumping factor
antigen.

Mab 107 (μg/mL)

| | | 2.5 | 2.5 | 2.5 | 2.5 |
|---|---|---|---|---|---|
| | RxClf (μg/mL) | 0.75 | 0.625 | 0.5 | 0.375 |
| ClfA | 250.00 | 0.700 | 0.661 | 0.593 | 0.483 |
| (pg/ml) | 125.00 | 0.388 | 0.360 | 0.320 | 0.273 |
| | 62.50 | 0.233 | 0.209 | 0.182 | 0.172 |
| | 31.25 | 0.152 | 0.144 | 0.131 | 0.107 |
| | 15.63 | 0.111 | 0.105 | 0.089 | 0.078 |
| | 7.81 | 0.089 | 0.079 | 0.068 | 0.062 |
| | 3.91 | 0.083 | 0.074 | 0.069 | 0.060 |
| | 0.00 | 0.071 | 0.075 | 0.068 | 0.058 |

The concentrations of each capping antibody are reported in μg/mL. The concentration of the antigen, Staphylococcal clumping factor, in each test is reported in pg/mL. The data are the the average absorbance readings at 405 nm for a minimum of at least two duplicate wells.

The combination of antibody concentrations selected to do limit of detection studies is shown in Table II-5.

TABLE II-5
Optimal antibody combinations for the detection of staphylococcal
antigens in a sample using an ELISA test.

| | | | MAb-107. | Affinity-purified |
|---|---|---|---|---|
| | MAb-76 | MAb 12.9 | biotin | RxClf40.biotin |
| Coating | 1 μg/mL | 7.5 μg/mL | N/A | N/A |
| concentration | | | | |
| Capping | N/A | N/A | 2.5 μg/mL | 0.75 μg/mL |
| concentration | | | | |

The optimal amounts of each antibody and the ratios for the respective capture and capping antibodies were determined from experiments using various combinations of antibody amounts and ratios. The criteria for selection of the optimal amount included the largest detection signal for each antigen, coupled with the lowest background signal when no antigen was present in the assay.

### Example II-2

### Determination of Lower Limit of Detection for Protein A and Clumping Factor Antigens in the Tandem Protein A/Clumping Factor Enzyme Linked Immunosorbant Assay (ELISA)

All procedures were performed as described in Example II-1. Antigens were prepared in filtered lysing solution—lysostaphin (Sigma Aldrich, St. Louis, Mo.) diluted to 3 μg/mL in antigen dilution buffer—PBS containing 0.2% w/v PLURONIC L64 (BASF, Florham Park, N.J.) and 50 mM disodium EDTA, pH 7.44.

Three antigens were run in the assay: 1) Clf40 clumping factor protein diluted by serial twofold dilutions from 2 ng/mL to 0.0019 ng/mL; 2) Zymed Protein A (SpA) diluted by serial twofold dilutions from 250 pg/mL to 0.24 pg/mL; and 3) S. aureus strain 25923, prepared as in Example II-1, except that it was diluted in serial two-fold dilutions to final sample concentrations of approximately 5×10⁵ CFU/mL to approximately 4.8×10² CFU/mL. No antigen control (lysing solution, as described above) was also included on the plates.

The lower limit of detection for each antigen was determined by choosing the sample concentration values that were three standard deviations above the no antigen control, with continually increasing absorbance values at that concentration point and above. For the tandem assay, the lower limit of detection for clumping factor antigen was less than or equal to 0.0019 ng/mL, and the lower limit of detection for protein A was 7.81 pg/mL. The lower limit of detection for lysed S. aureus strain 25923 was 3,828 cells/mL. The results of this study are shown in Table II-6.

TABLE II-6
Detection of staphylococcal antigens in an ELISA assay.
Capture Antibodies: Mab 12-9 (7.5 μg/mL) + MAb-76 (1 μg/mL)

| Clumping Factor | | Protein A | | Lysed S. aureus | |
|---|---|---|---|---|---|
| Clf | | SpA | | S. aureus | |
| (ng/mL) | Tandem | (pg/mL) | Tandem | (cfu/mL) | Tandem |
| 2.00000 | 4.679 | 250.00000 | 1.653 | 490,000 | 5.000 |
| 1.00000 | 3.022 | 125.00000 | 0.943 | 245,000 | 5.000 |
| 0.50000 | 1.789 | 62.50000 | 0.620 | 122,500 | 5.000 |
| 0.25000 | 1.065 | 31.25000 | 0.350 | 61,250 | 3.831 |
| 0.12500 | 0.646 | 15.62500 | 0.237 | 30,625 | 2.475 |
| 0.06250 | 0.430 | 7.81250 | 0.230 | 15,313 | 1.496 |
| 0.03125 | 0.321 | 3.90625 | 0.127 | 7,656 | 0.855 |
| 0.01563 | 0.276 | 1.95312 | 0.132 | 3,828 | 0.493 |
| 0.00781 | 0.247 | 0.97656 | 0.135 | 1,914 | 0.313 |
| 0.00391 | 0.242 | 0.48828 | 0.116 | 957 | 0.233 |
| 0.00195 | 0.234 | 0.24414 | 0.115 | 479 | 0.177 |
| 0.00000 | 0.221 | 0.00000 | 0.117 | 0 | 0.196 |
| 0 + 3 SD | 0.221 | 0 + 3 SD | 0.136 | 0 + 3 SD | 0.435 |

The values shown for the Tandem assays are the average absorbance (405 nm) readings from a minimum of at least two duplicate wells.

### Example II-3

### Determination of Lower Limit of Detection for Lysed and Non-Lysed Staphylococcus aureus Bacteria in the Tandem Protein A/Clumping Factor Enzyme Linked Immunosorbant Assay (ELISA)

All procedures were performed as described in Example II-1 except that, in this example, the S. aureus washed cells were split into two samples—lysed and unlysed. They lysed samples were treated as described in Example II-1. S. aureus strain 222 was a clinical isolate.

All antigens, with the exception of the unlysed S. aureus, and the standard curves were prepared in filtered lyse solution lysostaphin (Sigma Aldrich, St. Louis, Mo.) diluted to 3 μg/mL in antigen dilution buffer—PBS containing 0.2% w/v PLURONIC L64 (BASF, Florham Park, N.J.) and 50 mM disodium EDTA, pH 7.44. The unlysed S. aureus was prepared in the antigen dilution buffer described in Example II-1A.

Standard curves were prepared with Clf40 clumping factor protein and Zymed Protein A. Clf40 clumping factor protein was prepared in twofold serial dilutions from 2.5 ng/mL to 0.20 ng/mL. Protein A was prepared in twofold serial dilutions from 400 pg/mL to 3.13 pg/mL. A no antigen control was also included on the plate.

S. aureus strains 25923 and 222 were grown at 37° C. in tryptic soy broth (Hardy Diagnostics, Santa Maria, Calif.) overnight to an estimated concentration of 5.0×10⁸ cfu/mL. The cultures were washed twice—the broth cultures were centrifuged at 4° C. for 10 minutes at 10,000 rpm, then resuspended in PBSL (PBS containing 0.2% w/v PLURONIC L64.) Each washed bacterial stock was diluted in PBSL in serial tenfold dilutions to estimated concentrations of 1.0×10⁸ cfu/mL to 1.0×10³ cfu/mL (unlysed cells.) Aliquots of these diluted samples were then further diluted 1:10 into lyse solution, for final sample concentrations of 1.0×10⁷ cfu/mL to 1.0×10² cfu/mL (lysed cells), along with a zero bacteria control. Unlysed, diluted bacteria were plated on blood agar plates to verify the concentration of the original broth cultures. Assay results reflect the actual starting bacterial concentration.

Individual stabilized microtiter plates were washed and coated with one of the capture antibody solutions: MAb 12-9, MAb-76, or the mixture of MAb 12-9 and MAb-76. Antigen samples (serial dilutions of lysed S. aureus, serial dilutions of unlysed S. aureus, and the lyse solution control) were added to duplicate wells in each of the coated plates. Standard curve samples of clumping factor and Protein A were added to plates that were coated with capture antibodies to those respective antigens.

Three capping antibody preparations were made: 1) MAb-107.biotin at 2.5 μg/mL, 2) Affinity purified rabbit anti-clumping factor.biotin at 2.5 μg/mL, and 3) a tandem capping antibody mixture prepared by mixing an equal volume of MAb-107.biotin at 5 μg/mL with the affinity purified rabbit anti-clumping factor.biotin at 1.5 μg/mL, for final concentrations of 2.5 μg/mL and 0.75 μg/mL, respectively. The capping antibody mixtures were added to the appropriate microtiter plate and the ELISA assay was completed.

Representative results from the experiments are shown in Table II-7. The data from the Protein A plate and the tandem capture/capping plate showed more sensitive detection for lysed bacteria compared to whole bacteria. Results from the clumping factor plate showed equivalent detection for lysed and whole bacteria. Overall, the tandem coating/capture assay detected the lowest levels of bacteria on the standard curve when compared with the assays using single capture antibodies.

TABLE II-7
Detection of whole-cell and lysed Staphylococcus aureus using an optimized ELISA assay.

| Plate Specificity | Strain 25923 | Strain 25923 | Strain 222 | Strain 222 |
|---|---|---|---|---|
| (coating, capping Abs) | (lysed) | (unlysed) | (lysed) | (unlysed) |
| Protein A | 1 × 10⁷ cfu/mL | 1 × 10⁵ cfu/mL | 1 × 10⁶ cfu/mL | 1 × 10⁴ cfu/mL |
| (MAb-76, MAb-107) | | | | |
| Clumping Factor | 1 × 10⁶ cfu/mL | 1 × 10⁶ cfu/mL | 1 × 10⁶ cfu/mL | 1 × 10⁶ cfu/mL |
| (Mab 12-9, affinity-purified RxClf40) | | | | |
| Tandem | 1 × 10⁶ cfu/mL | 1 × 10⁴ cfu/mL | 1 × 10⁵ cfu/mL | 1 × 10⁴ cfu/mL |
| (tandem coat, tandem cap) | | | | |

The data show the lowest concentration of bacteria detected in at least duplicate wells in which each of the conditions was tested. The left column shows the antibodies used for the coating and capping reactions, respectively. The capping antibodies were biotinylated prior to use in the assay.

### Example II-4

### Determination of Inhibition of ELISA Activity of 3M S. aureus Strain 222 by Non-Neutralized N-acetyl L-Cysteine in Tandem Protein A/Clumping Factor Enzyme Linked Immunosorbant Assay (ELISA)

### A. General Materials and Methods

The antibodies used in the ELISA assay included two anti-Staphylococcus aureus protein A monoclonal antibodies (MAb-76, and MAb-107, the anti-Staphylococcus aureus clumping factor monoclonal antibody, MAb 12-9). Prior to use in the ELISA assays, the RxClf40 antibody was affinity purified as described in Example II-1. All capping antibodies were biotinylated prior to use. Antibodies were biotinylated according to the manufacturer's instructions using the EZ/Link NHS-PE04-Biotin kit from Pierce (Rockford, Ill.).

Phosphate buffered saline (PBS, 137 mM NaCl and 2.7 mM KCl in 10 mM phosphate buffer, pH 7.50) used in these experiments was prepared from a 10× concentrated solution obtained from EMD Biosciences (San Diego, Calif.). The PBST reagent used in the ELISA assay was prepared by adding 0.05% (v/v) TWEEN 20 to the PBS buffer. Costar 96-well high-binding polystyrene microtiter plates were obtained from Corning LifeSciences (Acton, Mass.). All buffers were filtered prior to use except the wash buffer. All ELISA wash procedures included five sequential wash volumes of 200 microliters per well and all washes were done with PBST buffer. Alkaline phosphatase chromogenic substrate, pNPP, was obtained from KPL (Gaithersburg, Md.).

The antigen used in these experiments was the cells of 3M S. aureus strain 222. To prepare the S. aureus antigen, strain 222 was grown at 37 C in tryptic soy broth (Hardy Diagnostics, Santa Maria, Calif.) overnight to an estimated concentration of 5.0×10⁸ cfu/mL. The culture was washed twice (centrifuged at 4 C for 10 minutes at 10,000 rpm), then resuspended in PBS. The washed bacterial stock was diluted to 2.0×10⁵ cfu/mL in PBS. This was further diluted 1:20 during antigen sample preparation for a final sample concentration of 1.0×10⁴ cfu/mL.

The N-acetyl L-Cysteine (NAC) used in these experiments was Sigma grade and was obtained from Sigma-Aldrich (St. Louis, Mo.). To prepare the non-neutralized NAC, NAC powder was added to PBS to make a 4% (w/v) solution, and then serial twofold dilutions were made in PBS to produce 2% and 1% solutions. To prepare the neutralized NAC, NAC powder sufficient for a 4% solution was added to a reduced volume of PBS. The pH was adjusted to near neutral pH with 5 M NaOH, and then additional PBS was added to bring the total volume to a level forming a 4% solution. Serial twofold dilutions were made in PBS to produce 2% and 1% solutions. PBS buffer without NAC was used as a 0% NAC control. The pH of each solution was measured, then each solution was filter sterilized with a 0.22 μM filter.

TABLE II-8
pH measurements of neutralized and non-neutralized
NAC solutions used in the study.

| | Neutralized | Non-neutralized |
|---|---|---|
| 4% | 7.18 | 1.58 |
| 2% | 7.26 | 1.79 |
| 1% | 7.35 | 2.05 |

The lysostaphin used in this study was obtained from Sigma-Aldrich. To prepare the lysostaphin, the lysostaphin stock was diluted in PBS to 200 μg/mL, then filter sterilized with a 0.22 μM filter. This was further diluted 1:20 during antigen sample preparation for a final sample concentration of 10 μg/mL.

All procedures were performed at room temperature unless specified otherwise.

### B. ELISA Assay Conditions

Two capture antibodies were used in the assay; MAb 76 and MAb 12-9. The antibodies were diluted from their refrigerated (4° C.) storage starting concentrations in PBS to 2.0 μg/mL for MAb 76 and 15 μg/mL for MAb 12-9; the two antibody solutions were then combined in equal volumes to the final microwell coating concentrations of 1.0 μg/mL for MAb 76 and 7.5 μg/mL for MAb 12-9. One hundred microliters of the coating solution were added to the wells of the microwell plate. The plate was incubated at 37° C. for 60 minutes. The coating solution was removed prior to the blocking step by washing.

The microtiter plate was blocked with 200 μL/well of StabilCoat immunoassay stabilizer (Surmodics, Eden Prairie, Minn.), then incubated overnight at 4° C. The blocking solution was removed prior to the antigen coating step by washing.

One hundred microliters of the solution of capture antigens were added to each well and the plate was incubated for 60 minutes at 37° C. The plate was washed subsequently with PBST solution.

Two capping antibodies were used in the assay; biotin-labeled MAb-107 and biotin-labeled RxClf40. The antibodies were diluted from their refrigerated (4° C.) storage starting concentrations in blotto (2% dried nonfat milk in PBST) to 5.0 μg/mL for MAb 107 and 1.5 μg/mL for RxClf40. The two antibody solutions were then combined in equal volumes to the final microwell coating concentrations of 2.5 μg/mL for MAb 107 and 0.75 μg/mL for RxClf40. One hundred microliters of the capping antibody mixture was added to each well and the plates were incubated for 60 minutes at 37° C.

Streptavidin-conjugated alkaline phosphatase (Streptavidin AP, Jackson ImmunoResearch Laboratories, Inc., West Grove, Pa.) was diluted in blotto to a concentration of 0.5 μg/mL. The microtiter plate was washed, and 100 μL/well of the Streptavidan.AP was added to the plate. The plate was incubated for 60 minutes at 37° C.

The plate was washed. Subsequently, 100 μL/well of pNPP substrate was added to the plate. The plate was held at room temperature for 15 minutes to observe the development of color. The alkaline phosphatase reaction was stopped by adding 100 μL/well of 5% (w/v) disodium EDTA and the plates were placed in a plate reader, where the absorbance at 405-nm wavelength was read.

C. Determination of Inhibition of ELISA Activity of 3M S. aureus Strain 222 by Non-Neutralized N-acetyl L-Cysteine in Tandem Protein A/Clumping Factor Enzyme Linked Immunosorbant Assay (ELISA)

ELISA assay was set up as above. The objective of this study was to determine if antigenic activity of 3M S. aureus strain 222 is inhibited by non-neutralized N-acetyl L-Cysteine (NAC). S. aureus cells, lysostaphin, and neutralized or non-neutralized NAC were combined in different sequences and were then used as the antigen in the tandem assay described above.

Sample preparation: NAC, S. aureus cells and lysostaphin were mixed in different sequences according to the procedures shown below, and then were immediately added to the microtiter plate in duplicate. This was repeated for each NAC solution tested.

1. No-bacteria control: Added 11.25 μL PBS and 11.25 μL lysostaphin to 202.5 μL NAC and vortexed for 30 seconds.

2. NAC+(S. aureus+lysostaphin): Mixed equal volumes of S. aureus stock and lysostaphin; and vortexed 30 seconds. Added 22.5 μL of this mixture to 202.5 μL of NAC and vortexed for an additional 30 seconds.

3. (NAC+S. aureus)+lysostaphin: Mixed 11.25 μL S. aureus stock and 202.5 μL of NAC and vortexed for 30 seconds. Added 11.25 μL lysostaphin and then vortexed for an additional 30 seconds.

4. (NAC+lysostaphin)+S. aureus: Mixed 11.25 μL lysostaphin and 202.5 μL of NAC and vortexed for 30 seconds. Added 11.25 μL S. aureus stock and vortexed for an additional 30 seconds.

Table II-9 shows ELISA results from the study. The data indicates that non-neutralized NAC eliminates any ELISA activity of S. aureus strain 222 in this assay, regardless of the solution strength or mixing order tested.

TABLE II-9
ELISA Study results

| | Sample preparation | 4% | 2% | 1% |
|---|---|---|---|---|
| | procedure | NAC | NAC | NAC |
| Neutralized NAC | 1. No bacteria control | 0.097 | 0.1155 | 0.119 |
| | 2. NAC + (S. aureus + | 0.7785 | 0.697 | 0.6815 |
| | lysostaphin) | | | |
| | 3. (NAC + S. aureus) + | 0.9475 | 0.68 | 0.7625 |
| | lysostaphin | | | |
| | 4. (NAC + lysostaphin) + | 0.838 | 0.8585 | 0.9795 |
| | S. aureus | | | |
| Non-neutralized | 1. No bacteria control | 0.0785 | 0.084 | 0.087 |
| NAC | 2. (NAC + lysostaphin) + | 0.0785 | 0.0775 | 0.0845 |
| | S. aureus | | | |
| | 3. (NAC + S. aureus) + | 0.066 | 0.072 | 0.0805 |
| | lysostaphin | | | |
| | 4. NAC + (S. aureus + | 0.0765 | 0.07 | 0.088 |
| | lysostaphin) | | | |

0% NAC positive control: [PBS + (S. aureus + lysostaphin)]: 0.684
Additional controls:
Lysostaphin stock solution: 0.095
PBS diluent: 0.0925

### III. Methods of Analyzing Samples for Bacteria Using Acoustic Wave Sensors

### Example III-1

### Conjugation of Mab107-Biotin to Magnetic Particles

Mab 107 IgG, Mab 76 IgG, Rabbit anti-Clf40 IgG, Goat anti-Clf40 IgG, Mab 12-9 IgG (Inhjbitex, Alpharetta, Ga.) are biotinylated using the EZ-Link NHS-PEO4-Biotin kit (Pierce, Rockford, Ill.) according to the manufacturer's instructions. Protein A is obtained from Invitrogen and diluted in PBS to the desired test concentration. Streptavidin-coated magnetic particles and biotinylated Mab 107 IgG, Mab 76 IgG, Rabbit anti-Clf40 IgG, Goat anti-Clf40 IgG, Mab 12-9 IgG antibody combinations (two or more) are mixed together at the desired concentrations and incubated at 37° C. for at least 1 hr in PBSL buffer. The sample is then washed three times in PBSL buffer to remove any unbound antibody. After the final wash, the supernatant is replaced with 1 mL of the Protein A test sample in PBSL buffer, and incubated at 37° C. for at least 30 minutes.

### Example III-2

### Methods of Preparing Sensors and Running Detection Experiments

A shear-horizontal surface acoustic wave (SH-SAW) sensor (supplied by Com Dev (Cambridge, Ontario, Canada) or by Sandia National Laboratory (Albuquerque, N. Mex.)) spin coated with a waveguide (50:50 copolymer of methyl methacrylate and isobornyl methacrylate prepared as described in Example W1 of PCT Publication No. WO2005/066092 titled “Acoustic Sensors and Methods”, filed on Dec. 17, 2004) are used in the experiments. The sensors are sprayjet-coated with an immobilization chemistry comprising a terpolymer of iso-bornyl methacrylate/methyl methacrylate/Saccharin-methacrylate/acryloyloxybenzophenone 35/35/30/0.5 made in Butyl acetate/Acetonitrile 50/50 prepared as described in Example MP26 of PCT Publication No. WO2005/066092 titled “Acoustic Sensors and Methods”, filed on Dec. 17, 2004.

In some cases, a monoclonal antibody (Mab107) specific to Protein A, Mab 76 IgG specific to Protein A, Rabbit anti-Clf40 IgG, Goat anti-Clf40 IgG, Mab 12-9 IgG (Clumping Factor protein-specific; Inhibitex, Alpharetta, Ga.) in combinations of 2 or more antibodies are hand-coated or sprayjet-coated on both (active and reference) sensor channels. In other cases, the Mab 107 IgG, Mab 76 IgG, Rabbit anti-Clf40 IgG, Goat anti-Clf40 IgG, Mab 12-9 IgG (Inhibitex, Alpharetta, Ga.) or the Rabbit anti-staph aureus (RaSa) (Accurate Chemical & Scientific Corporation, Westbury, N.Y.) in combinations of two or more antibodies are hand-coated or sprayjet-coated on one sensor channel (Active channel) and a nonspecific Chicken IgY (Jackson ImmunoResearch Laboratories Inc, West Grove, Pa.) is hand-coated or sprayjet-coated on the other channel (Reference channel).

The coated sensor is heat-bonded to a flexible circuit via conductive adhesive (Anisotropic conductive film adhesive 7313, 3M Company, St. Paul, Minn.). The bonded sensor is attached to a temperature-controlled flowpod via double-sided adhesive film. The sensor is then connected to an electronic measurement board (via the flex circuit) driven by a software program written in LabVIEW software using a network analyzer. LabVIEW software can be obtained from National Instruments (Austin, Tex.). Attenuation and phase properties can be collected throughout the experiment in the desired frequency range.

To start the experiment, PBSL running buffer (described below) is flowed over the sensor at an average flow rate of 0.1 mL/min via a syringe pump and then adjusted to the desired flow rate. The software program is then used to initiate the experiment. A rare earth magnet composed of Neodymium-Iron-Boron (NdFeB) is raised into position underneath the sensor. After sufficient flow stabilization, the sample is injected via an injection valve and flowed over the sensor at a time specified by the software.

After the sample reaches and is collected on the sensor surface, the magnet is moved (“dropped”) a sufficient distance (at a time specified by the software) to significantly reduce the magnetic field strength at the sensor surface. Typically the magnet is moved greater than 65 mm. However, the field strength is significantly reduced at much smaller distances.

Flow is continued a sufficient time until the phase and attenuation signals are stabilized. (Typically, this is determined by visual inspection of the phase and attenuation raw signals that are displayed on the computer screen. When the changes in the raw signal over time are relatively small compared to the signal changes expected after the magnet is dropped, the signals are considered to be stable.).

A time gating algorithm (Page 3-35 and 3-36 in 8753ET/ES Network Analyzers User's Guide, Agilent Technologies) is used to process the raw phase and attenuation data generated from the experiment. Unless specified otherwise, the time interval unit for data collection is 13 seconds and the time commenced when the data collection is started by the software. Appropriate gates for the algorithm may be specified based on the specific sensor design that is being used. The algorithm can be applied directly through the network analyzer such that the data obtained from the experiment is already time gated. Alternatively, the raw data can be collected and time gating can be done using a software program written in Matlab (The Mathworks, Natick, Mass.).

The time gated data are further analyzed to determine shifts in phase and attenuation. All of this data processing is done using the Matlab software. For those cases where there is no reference channel, the shift in the signal for both phase and attenuation in the two channels is computed by subtracting its value just before the magnet is dropped from its value when the signal has stabilized after the magnet is dropped.

For sensors with both an active and reference channel, a difference signal is calculated by subtracting the attenuation and phase signal of the reference channel from that of the active channel, respectively. The shift in this difference signal is computed by subtracting the value just before the magnet is dropped from the stable signal obtained after the magnet is dropped.

The complete disclosures of all patents, patent applications, publications, and nucleic acid and protein database entries, including for example GenBank accession numbers, that are cited herein are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

1. A method of capturing an analyte characteristic of a specific bacterium, the method comprising:
providing a sample suspected of including target whole cells comprising one or more analytes characteristic of a specific bacterium;
providing two or more antibodies having antigenic specificities for two or more distinct analytes characteristic of the specific bacterium, wherein the antibodies comprise at least one monoclonal antibody;
providing a solid support material; and
providing contact between the sample, the solid support material, and the two or more antibodies under conditions effective to capture target whole cells with one or more analytes characteristic of a specific bacterium, if present.

2. The method of claim 1, wherein the two or more antibodies are attached to the solid support material forming an analyte-binding material, and the method includes providing contact between the sample and the analyte-binding material under conditions effective to capture whole cells with one or more analytes characteristic of a specific bacterium, if present.

3. The method of claim 2, wherein providing contact between the sample and the analyte-binding material comprises simultaneous contact between the sample and the two or more antibodies.

4. The method of claim 1, wherein providing contact between the sample, the solid support material, and the two or more antibodies comprises providing contact between the two or more antibodies and the sample to form antibody-bound whole cells, and subsequently providing contact between the antibody-bound whole cells and the solid support material.

5-7. (canceled)

8. The method of claim 1, wherein the antibodies are selected from the group consisting of MAb-76, MAb-107, affinity-purified RxClf40, affinity-purified GxClf40, MAb 12-9, fragments thereof, and combinations thereof.

9-11. (canceled)

12. The method of claim 1, wherein at least 20% of the target whole cells are captured.

13. The method of claim 12, wherein at least 50% of the target whole cells are captured.

14. (canceled)

15. The method of claim 1, wherein the solid support material comprises particles at a concentration of greater than 0.04 mg/mL.

16. The method of claim 1, wherein the solid support material comprises particles and the antibody to particle ratio is greater than 1 μg/mg particles.

17. The method of claim 1, wherein the solid support material comprises particles and the antibody to particle ratio is at least 0.01 μg/mg particles.

18. The method of claim 17, wherein the antibody to particle ratio is less than 10 μg/mg particles.

19. The method of claim 1, wherein the solid support material comprises particles and each particle has at least two antibodies that bind different analytes disposed thereon.

20. A method of analyzing a sample for a bacterium, the method comprising:
providing a sample suspected of including one or more analytes characteristic of a specific bacterium;
providing two or more immobilized antibodies having antigenic specificities for two or more distinct analytes characteristic of the specific bacterium;
providing two or more labeled antibodies having antigenic specificities for two or more distinct analytes characteristic of the specific bacterium, wherein the antibodies are labeled with a direct or indirect enzymatic label;
providing contact between the sample, the immobilized antibodies, and the labeled antibodies to bind the one or more analytes between the labeled antibodies and the immobilized antibodies;
wherein, for each of the analytes present, the immobilized antibodies and the labeled antibodies comprise two or more antigen-binding pairs; and
analyzing for the presence or absence of the specific bacterium.

21. The method of claim 20, wherein providing contact between the sample, the immobilized antibodies, and the labeled antibodies comprise:
contacting the sample with the immobilized antibodies under conditions effective to capture one or more analytes characteristic of a specific bacterium, if present in the sample, to form one or more captured analytes; and
contacting the one or more captured analytes, if present, with the labeled antibodies under conditions effective to cause binding between the one or more captured analytes and the labeled antibodies.

22. The method of claim 21, wherein contacting the sample with the immobilized antibodies comprises providing contact between the sample and each immobilized antibody simultaneously.

23. The method of claim 21, wherein contacting the one or more captured analytes, if present, with the labeled antibodies comprises providing contact between the captured analytes and each labeled antibody simultaneously.

24. The method of claim 20, wherein providing contact between the sample, the immobilized antibodies, and the labeled antibodies comprises;
contacting the sample with the labeled antibodies under conditions effective to cause interaction between the one or more analytes characteristic of a specific bacterium, if present in the sample, and the labeled antibodies; and
contacting the immobilized antibodies with the sample containing the labeled antibodies under conditions effective to cause binding between the labeled antibodies, the one or more analytes, and the immobilized antibodies.

25. (canceled)

26. The method of claim 20, wherein the antibodies are selected from the group consisting of MAb-76, MAb-107, affinity-purified RxClf40, affinity-purified GxClf40, MAb 12-9, fragments thereof, and combinations thereof.

27. The method of claim 20, wherein the immobilized antibodies are hound to a solid support material.

28. The method of claim 27, wherein the solid support material comprises the surface of a microwell plate.

29. The method of claim 28, wherein a well of the microwell plate has immobilized therein a mixture of antibodies.

30. (canceled)

31. The method of claim 29, wherein the mixture of antibodies comprises a polyclonal antibody and a monoclonal antibody.

32. The method of claim 20, wherein the immobilized antibodies comprise MAb12-9 and MAb-76.

33. The method of claim 20, wherein the labeled antibodies comprise antibodies MAb-107 and affinity-purified RxClf40.

34. The method of claim 20, wherein the labeled antibodies comprise a direct enzymatic label.

35. The method of claim 20, wherein the labeled antibodies comprise biotin linked through a polyethylene oxide linker.

36. The method of claim 20, wherein if the labeled antibodies comprise an indirect label, the method further includes reacting the labeled antibodies with an enzyme conjugate before, during, or after contacting the one or more captured analytes, if present, with the labeled antibodies.

37. The method of claim 20, wherein analyzing for the presence or absence of the specific bacterium comprises analyzing colorimetrically.

38. The method of claim 37, wherein the colorimetric analysis comprises using a chromogenic enzyme substrate.

39-41. (canceled)

42. The method of claim 20, wherein the sample comprises lysed cells.

43. (canceled)

44. The method of claim 20, wherein analyzing for the presence or absence of the specific bacterium comprises quantifying the total amount of analyte present.

45. The method of claim 20, wherein the sample comprises whole cells.

46. (canceled)

47. A method of analyzing a sample for a bacterium, the method comprising:
providing a sample suspected of including one or more analytes characteristic of a specific bacterium;
providing two or more particle—antibody conjugates having antigenic specificities for two or more distinct analytes characteristic of the specific bacterium;
providing a system comprising an acousto-mechanical sensor comprising a detection surface comprising two or more immobilized antibodies having antigenic specificities for two or more distinct analytes characteristic of the specific bacterium;
providing contact between the sample, the immobilized antibodies on the detection surface of the acousto-mechanical sensor, and the particle-antibody conjugates to bind the one or more analytes between the particle-antibody conjugates and the immobilized antibodies;
wherein, for each of the analytes present, the immobilized antibodies and the particle-antibody conjugates comprise two or more antigen-binding pairs; and
analyzing for the presence or absence of the specific bacterium.

48. The method of claim 47, wherein providing contact between the sample, the immobilized antibodies on the detection surface of the acousto-mechanical sensor, and the particle-antibody conjugates comprises:
contacting the sample with the particle-antibody conjugates, under conditions effective to cause interaction between the one or more analytes characteristic of the specific bacterium, if present in the sample, and the particle-antibody conjugates; and
contacting the detection surface of the acousto-mechanical sensor with the sample containing the particle-antibody conjugates under conditions effective to cause binding between the particle-antibody conjugates, the one or more analytes, and the immobilized antibodies.

49. The method of claim 47, wherein providing contact between the sample, the immobilized antibodies on the detection surface of the acousto-mechanical sensor, and the particle-antibody conjugates comprises:
contacting the sample with the immobilized antibodies under conditions effective to capture one or more analytes characteristic of a specific bacterium, if present in the sample, to form one or more captured analytes; and
contacting the one or more captured analytes, if present, with the particle-antibody conjugates under conditions effective to cause binding between the one or more captured analytes and the particle-antibody conjugates.

50. The method of claim 49, wherein contacting the sample with the immobilized antibodies comprises providing contact between the sample and each immobilized antibody simultaneously.

51. The method of claim 49, wherein contacting the one or more captured analytes, if present, with the particle-antibody conjugates comprises providing contact between the captured analytes and each particle-antibody conjugates simultaneously.

52. The method of claim 47, wherein the particles of the particle-antibody conjugates comprise magnetic particles.

53. The method of claim 52, wherein each particle has at least two antibodies that bind different analytes disposed thereon.

54. The method of claim 52, wherein the method further comprises:
providing a magnetic field generator capable of providing a magnetic field proximate the detection surface that draws the target analyte with the attached magnetic particles to the detection surface of the sensor;
selectively attaching the target biological analyte with the attached magnetic particles to the detection surface;
disabling the magnetic field generator to substantially reduce the magnetic field proximate the detection surface; and
operating the acousto-mechanical sensor to detect the attached target biological analyte while the detection surface is submersed in liquid.

55. The method of claim 54, wherein the disabling of the magnetic field generator comprises removing the magnetic field generator a sufficient distance to substantially reduce the magnetic field proximate the detection surface.

56. The method of claim 47, wherein the acousto-mechanical sensor comprises a surface acoustic wave sensor.

57. The method of claim 56, wherein the surface acoustic wave sensor comprises a shear horizontal surface acoustic wave sensor.

58. (canceled)

59. The method of claim 47, wherein the antibodies are selected from the group consisting of MAb-76, MAb-107, affinity-purified RxClf40, affinity-purified GxClf40, MAb 12-9, fragments thereof, and combinations thereof.

60-61. (canceled)

62. The method of claim 47, wherein the sample comprises lysed cells.

63. (canceled)

64. The method of claim 47, wherein analyzing for the presence or absence of the specific bacterium comprises quantifying the total amount of analyte present.

65. (canceled)

66. An analyte-binding material comprising:
a solid support material;
antibodies MAb-76, MAb-107, affinity-purified RxClf40, affinity-purified GxClf40, MAb 12-9, fragments thereof, or combinations thereof, disposed on the solid support; and
optionally a detectable marker.

67-71. (canceled)
